(19) United States
(12) Patent Application Publication
(10) Pub. No.: US 2009/0259167 A1
(43) Pub. Date: Oct. 15, 2009

(54) METHODS AND COMPOSITIONS FOR DOSE-DEPENDENT PHOTODYNAMIC THERAPY OF DISORDERS

(75) Inventors: Fernanda H. Sakamoto, Boston, MA (US); R. R. Anderson, Boston, MA (US); Zeina Tannous, Boston, MA (US); William A. Farinelli, Danvers, MA (US); Apostolos G. Doukas, Belmont, MA (US)

Correspondence Address: EWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/409,150

(22) Filed: Mar. 23, 2009

Related U.S. Application Data
(60) Provisional application No. 61/042,641, filed on Apr. 4, 2008.
(60) Provisional application No. 61/044,460, filed on Apr. 11, 2008.

(51) Int. Cl. A61N 5/06; A61K 31/197; A61P 35/00

(52) U.S. Cl. 604/21; 604/20; 514/561

(57) ABSTRACT

The invention provides methods and compositions for treating a tissue disorder in a subject by parenterally administering a solution of aminolevulinic acid (ALA) or a derivative thereof that is not greater than 1.0 percent by weight into a local subcutaneous or dermal region of the subject; and administering high fluence light to said bodily area to produce a phototoxic species in said local region, thereby treating a tissue disorder in the subject.

### CROSS-REFERENCE TO RELATED APPLICATIONS

This application claims the benefit under 35 U.S.C. § 119(e) of makes reference to U.S. provisional application Ser. No. 61/042,641, filed Apr. 4, 2008, and U.S. provisional application Ser. No. 61/044,460, filed Apr. 11, 2008, the entire contents of both of which applications are incorporated herein by reference.

### BACKGROUND OF THE INVENTION

Photodynamic therapy is a process whereby light of a specific wavelength is directed to tissues undergoing treatment or investigation that have been rendered photosensitive through the administration of a photoreactive or photosensitizing agent. The objective of the intervention may be diagnostic, whereby the light is selected to cause the photoreactive agent to fluoresce, thus yielding information about the tissue without causing damage to the tissue. The objective may also be therapeutic, whereby the wavelength of light delivered to the photosensitive tissue under treatment causes the photoreactive agent to undergo a photochemical interaction with oxygen in the tissue under treatment which yields free radical species, such as singlet oxygen, which, in turn, cause local tissue destruction or cellular apoptosis.

Photodynamic therapy (PDT) has proven to be very effective in destroying abnormal tissues, such as cancer cells. Generally, PDT involves administering a photoreactive agent having characteristic light absorption properties to a patient. Abnormal tissue in the body is known to selectively absorb certain photoreactive agents to a much greater extent than normal tissue, e.g., tumors of the pancreas and colon may absorb two to three times the volume of these agents, compared to normal tissue. Even more effective selectivity can be achieved using a photoreactive agent that is bound to an antibody or targeting ligand, which links with antigens or molecular targets on a desired cell or tissue of interest.

Once the abnormal tissue has absorbed or linked with the photoreactive agent, the abnormal tissue can then be destroyed by administering light of an appropriate wavelength or waveband corresponding to the absorption characteristics of the photoreactive agent. To administer PDT to internal sites that are not accessible through a natural body orifice, a fiber optic probe (or other means for delivering light) can be inserted either through a needle or through a surgically created opening. When the internal treatment site is accessible through natural body orifices, an endoscope can be used to visualize the lesion and accurately direct and administer the light therapy to the treatment site.

There are two main types of photoreactive agents in clinical use for PDT therapy at present. The first type, methoxypsoralens, are given systemically. Ultraviolet light is used to activate them. Localized exposure of psoralen-containing tissues to ultraviolet light induces a localized photochemical reaction that causes the drug to bind covalently to the DNA of living cells, thus destroying their proliferative potential. The second type, porphyrins and related photoreactive agents, are also given systemically (by intravenous injection), but can also be administered topically or by intralesional injection. They can be activated by visible light. The localized exposure of porphyrin-containing tissues to such light ordinarily does not induce a chemical reaction between cell components and the porphyrin molecules. Instead, the porphyrins act as catalysts by trapping the energy of the photoactivating light and then passing it on to molecules of oxygen, which, in turn, are raised to an excited state that is capable of oxidizing adjacent molecules or structures. Cell death is not caused primarily by damage to the DNA, but by damage to essential membrane structures.

One particular use of PDT in recent times has been in the dermatological area and has involved therapies for treating various skin disorders, as well as for the reduction of skin-related tissue or cells, such as, adipose tissue. Dermatological use of PDT is generally performed by first administering a photosensitive compound systemically or topically, followed by illuminating the treatment site at a wavelength or waveband which closely matches the absorption spectra of the photosensitizer. In doing so, singlet oxygen and other reactive species are generated leading to a number of biological effects which result in cytotoxicity. However, the depth and volume of the cytotoxic effect in skin tissue can depend on a complex interaction of light penetration in tissue, the photosensitizer concentration and cellular location, and availability of molecular oxygen. In general, topical use of photoreactive agents for PDT of skin-related conditions is severely limited by the level of drug uptake and light penetration. Moreover, current PDT methods are not capable of easily targeting PDT therapy to specific regions or microanatomical structures of the skin, such as the different skin layers, different subcutaneous structures and glands, adipose tissue, or skin-related diseased tissues.

There continues to be a need in the art for improved, more effective and more selective methods of PDT that are capable of easily and effectively treating skin disorders and diseases, including skin-associated adipose tissue, and which are relatively free of harmful or undesirable side-effects typically associated with PDT therapy.

### SUMMARY OF THE INVENTION

The present invention provides methods and compositions for treating and/or diagnosing various tissue disorders in a subject by photodynamic therapy. For example, the invention provides intradermal ALA photodynamic therapy for variety of skin disorders, such as vascular lesions (e.g., malformations, hemangiomas, tumors) and eccrine gland disorders. In addition, the photodynamic therapy methods of the invention can be utilized for removal of fat tissue or cells. Further, the invention relates to specific dose-dependent targeting of skin structures (e.g., non-epidermal skin structures) using ALA photodynamic therapy. The invention makes use of very low concentration (e.g., ≦about 0.016%) of ALA solutions targeted fat, moderate doses of ALA concentration (e.g., ˜0.06%) selectively targeted eccrine glands and ALA concentrations ≧about 0.25% provide a potent vascular PDT reaction. In particular, the invention provides for the administration of a solution of a photosensitizer, e.g., a photosensitizer precursor drug, such as aminolevulinic acid (ALA), and derivatives thereof, into the skin of the subject followed by illumination with high-fluence light, e.g., red light, to yield a photodynamic effect.

Thus, in one aspect, the invention provides a method for treating a tissue disorder in a subject, the method comprising the steps of: parenterally administering a solution of aminolevulinic acid (ALA) or a derivative thereof that is not greater than 1.0 percent by weight into a bodily area of the subject; administering high fluence red light to said bodily area to produce a phototoxic species in said local region, thereby treating a tissue disorder in the subject. Preferably, the bodily area is a local subcutaneous or dermal region of the subject. Preferably still, the bodily area excludes the epidermis.

In another aspect, the present invention provides a method for treating a tissue disorder in a subject, the method comprising the steps of: parenterally administering a solution comprising about 1.0 percent or less than 1.0 percent by weight aminolevulinic acid (ALA) or a derivative thereof and at least one antinociceptive agent into a bodily area of the subject; administering high fluence red light to said bodily area to produce a phototoxic species in said local region, thereby treating a tissue disorder in the subject. Preferably, the bodily area is a local subcutaneous or dermal region of the subject. Preferably still, the bodily area excludes the epidermis.

In still another aspect, the present invention provides a method for reduction of lipid rich cells in a bodily area of a subject, the method comprising the steps of: providing a solution of aminolevulinic acid (ALA) or a derivative thereof that is not greater than 1.0 percent by weight to a bodily area comprising fat tissue of the subject; administering high fluence red light to said bodily area to produce a phototoxic species in said local region, thereby reducing lipid rich cells in the subcutaneous tissue of a subject. Preferably, the bodily area is a local subcutaneous or dermal region of the subject. Preferably still, the bodily area excludes the epidermis.

In certain embodiments, the solution is provided by parenteral administration, for example, by injection, or in particular, by intradermal injection, subcutaneous injection, intraperitoneal injection or intravenous injection.

In other embodiments, the tissue disorder is a vascular malformation, such as a hemangioma, a Port wine stain, Sturge-Weber Syndrome, or a secondary neovascularization. In certain other aspects, the tissue disorder is non-neoplastic. The tissue disorder can also be a tumor or an eccrine gland disorder, such as hyperhidrosis, hidradenitis suppurativa, syringomas, or bromohydrosis.

In a certain embodiments, the wavelength of the red light is between about 620 and about 645 nm. The fluence of the red light can be between about 100 and about 300 J/cm² The irradiance of the red light can be between about 30 and about 200 mW/cm².

In other embodiments, the aminolevulinic acid derivative is an ester of 5-aminolevulinic acid.

In another embodiment, the solution of the aminolevulinic acid is between about 0.015% and about 1.0% ALA. The solution, in another embodiment, can be between about 0.0005% and about 1.0% ALA, or it can be about 0.06% ALA or about 0.25% ALA. In one particular embodiment, the solution is less than or equal to about 0.016% ALA.

In another embodiment, the bodily area is a local subcutaneous or dermal region. The bodily area can also comprise the epidermis, dermis, or subcutis layer of skin. In other embodiments, the bodily area is an internal body organ or tissue. In still other embodiments, the bodily area comprises a local subcutaneous region of the subject.

In certain embodiments, the fat tissue is part of an internal organ or tissue. The fat tissue can be part of the endovascular system or part of an atherosclerotic plaque of a blood vessel.

In one embodiment, the phototoxic species is a porphyrin. The porphyrin can be a protoporphyrin IX or a derivative thereof.

In a further embodiment, the solution can further comprise at least one antinociceptive agent, such as an opioid analgesic or a local anesthetic.

In another embodiment, the solution comprises about 1.0 percent by weight aminolevulinic acid (ALA) or a derivative thereof and at least one antinociceptive agent.

In another aspect, the invention provides a pharmaceutical composition comprising about 1.0 percent by weight aminolevulinic acid (ALA) or a derivative thereof and at least one antinociceptive agent. In one embodiment, the antinociceptive agent is an opioid analgesic. In another embodiment, the at least one antinociceptive agent is a local anesthetic.

In still another aspect, the invention provides a packaged pharmaceutical comprising aminolevulinic acid (ALA) or a derivative thereof that is not greater than 1.0 percent by weight and instructions for use in accordance with a method of the invention.

In one embodiment, the packaged pharmaceutical of the invention includes at least one antinociceptive agent. In another embodiment, the antinociceptive agent is an opioid analgesic or an anesthetic.

Other aspects of the invention are described in the following disclosure, and are within the ambit of the invention.

### BRIEF DESCRIPTION OF THE DRAWINGS

The following description, given by way of example, but not intended to limit the invention to specific embodiments described, may be understood in conjunction with the accompanying drawings, incorporated herein by reference. Various features and embodiments of the present invention will now be described by way of non-limiting example and with reference to the accompanying drawings in which:

FIG. 1. (A). Top images show fluorescence pictures from 1.000% to 0.0005% ALA solutions after 3 hours of incubation. Fluorescence was observed clinically down to 0.004%. Bottom images show clinical pictures^{§} of corresponding solutions 24 hours after PDT. Clinical reaction was present down to 0.016% solution showing mild erythema near the injected sites, although pinpoint erythematous spots at the needle puncture sites were observed for all concentrations. (B) Top images show fluorescence after 3 hours of incubation of topical/injected of reference sites that underwent different exposures: 20% topical ALA (with and without light exposure); 0.5% injected ALA without light exposure; light only and control sites. Bottom images show corresponding clinical responses after 24 hours.
^{§}Clinical photographs in the 0.016% to 0.0005% ALA sites were color balanced to photographs taken from 1.0% to 0.03% in a different animal. These image modifications minimized relative photographic flash exposure differences that varied from one animal to the other, without substantial changes in clinical response appearance.

FIG. 2. Fluorescence emission produced after 3 hours of incubation of different ALA injected solutions (1.0-0.0005%) before and after PDT compared to fluorescence produced by a 20% topical solution, and light only site and control site. Data obtained from pixel value analysis of digital fluorescent photographs as described.

FIG. 3. Fluorescence microscopy at 10× of skin structures (epidermis, hair follicle, eccrine and apocrine glands and fat) comparing porphyrin fluorescence produced by different injected ALA solutions, 20% topical ALA and control sites. Negative fluorescence is shown in black and white color represents maximum porphyrin accumulation. FIG. 3a shows 20% ALA applied topically and 1% to 0.03% injected solutions. FIG. 3b shows 0.01% to 0.0005% injected ALA solutions and control sites.

FIG. 4. Histopathology (H&E stain) slides 24 hours after red light exposure (PDT). 4a. Epidermis showing no relevant changes and superficial vessel inflammation with blood stagnation after 0.5% injected ALA-PDT (10×). 4b. Non-selective destruction of hair follicle, adnexal glands, and fat (10×) after 0.5% injected ALA-PDT. 4c. Selective reaction of eccrine glands after 0.06% injected ALA-PDT (10×). 4d. Massive fat septae necrosis after 0.002% injected ALA-PDT (5×). Abbreviations: HF: hair follicle; EG: eccrine glands.

FIG. 5. Summary of mean rates given by blind assessment by dermapathologist, of inflammatory reaction observed in different skin structures after 24 hours of injected ALA-PDT. Inflammatory responses in the epidermis, dermis, hair follicles, sebaceous glands, eccrine and appocrine glands, fat and muscle were rated on a scale from 0 to 3, where 0 represents no reaction; 1, mild reaction; 2, moderate reaction; 3, severe reaction (associated with necrosis and apoptosis).

### DETAILED DESCRIPTION OF THE INVENTION

The present invention generally provides methods and compositions for treating and/or diagnosing a tissue disorder, in particular, a skin-related disorder, in a subject using photodynamic therapy. In one aspect, the invention provides methods and compositions for photodynamic therapy of skin-related disorders, such as vascular lesions (e.g., malformations, hemangiomas, and tumors), gland disorders (e.g., eccrine gland disorders) and unwanted subcutaneous adipose tissue, utilizing photosensitive agents that, in a dose-dependent manner, specifically target the diseased cells or tissues. The invention is based, at least in part, on the discovery that certain photosensitive agents, when administered intradermally at particular depths, target different microscopic tissue structures, e.g., dermatological structures or sites, depending on the concentration or amount of the photosensitive agent administered. Because singlet oxygen and radical intermediates generated during PDT are highly reactive and cause oxidative damage precisely at the sites where they are generated, the methods and compositions of the invention provide an easy and effective clinical approach for targeting therapy and/or diagnosis of a specific tissue site or structure.

In certain aspects, the present invention provides compositions and methods for administering a photosensitive agent, such as aminolevulinic acid (ALA), into a bodily area of a subject, e.g., the skin of the subject, in particular, the dermis and subcutaneous layer of the skin, in a dose-dependent manner, followed by illumination with a suitable light source to yield a photodynamic effect on a specific skin-related tissue or component, such as, a skin-related gland, diseased site, or adipose tissue. In one aspect, the use of relatively low concentrations (as defined herein) of administered photosensitive agents of the invention are selectively targeted to adipose tissue, e.g., in a subcutaneous region, the use of relatively moderate doses (as defined herein) of administered photosensitive agents are selectively targeted eccrine glands and relatively high doses (as defined herein) of administered photosensitive agents of the invention are selectively targeted to the vasculature.

Terms, as used herein, are based upon their art recognized meaning and from the present disclosure and should be clearly understood by the ordinary skilled artisan. For the sake of clarity, terms may also have a particular meaning as would be clear from their use in context.

### DEFINITIONS

As used herein, the term “bodily area” refers to any site within the body, including any external or internal body surface, tissue, region or organ, which can be treated by the methods of the invention. The term encompasses the dermis and subcutaneous (including subcutaneous adipose tissue) regions of the skin, as well as any internal site of a body, including any organ, any bodily orifice, and any internal tissue (e.g., inner adipose tissue), including a blood vessel.

As used herein, the term “skin-related disorder” refers to any disorder affecting or relating to the skin or any skin component, such as a cell or tissue of the epidermis, dermis or subcutaneous layers, including any vascular cell or structure, nerve cell or structure, muscle cell or structure, adipose cell or structure, or gland, hair or nail component. The term “skin” encompasses any component or feature of the integumentary system, as well as adipose tissue associated therewith.

As used herein, the term “target disorder” encompasses skin-related disorders as defined herein, but also extends to any bodily area or site-internal or external-comprising a target or diseased tissue of interest. The term, target disorder, encompasses unwanted adipose tissue occurring anywhere in the body, including adipose tissue associated with organs, bodily cavities, abdomen, or even blood vessels.

As used herein, the term “skin-related structure” or the like refers to any anatomical structure of the skin, including the dermis and subcutaneous tissues of the skin, as well as any adipose tissue or cells in contact with or forming a part of the subcutaneous tissue. A skin-related structure can also include any skin-related components, such as hair, nails, glands skin-related vasculature (arteries, veins, and lymph vessels), and skin-related nerve fibers.

The structure of skin is well known. It will be appreciated that skin is composed of three primary layers: the epidermis, which provides waterproofing and serves as a barrier to infection; the dermis, which serves as a location for the appendages of skin (e.g., hair or glands); and the hypodermis (or subcutaneous adipose layer).

The epidermis is the outermost layer of the skin. It forms the waterproof, protective wrap over the body's surface and is made up of stratified squamous epithelium with an underlying basal lamina. The epidermis can be further subdivided into the following layers (beginning with the outermost layer): stratum, corneum, lucidum (e.g., palms of hands and bottoms of feet), granulosum, spinosum, and basale. Cells are formed through mitosis at the basale layer. The daughter cells move up the strata changing shape and composition as they die due to isolation from their blood source.

The epidermis is divided into five layers. The stratum corneum (“horny layer”) is the outermost layer of the epidermis and the skin. It is composed mainly of dead cells that lack nuclei. As these dead cells slough off, they are continuously replaced by new cells from the stratum germinativum (basale).

The stratum lucidum is a thin, clear layer of dead skin cells in the epidermis. It contains a clear substance called eleidin, which eventually becomes keratin. This layer is found beneath the stratum corneum of thick skin, and as such is only found on the palms of the hands and the soles of the feet. The keratinocytes of the stratum lucidum do not feature distinct boundaries and are filled with eleidin, an intermediate form of keratin.

The next layer is the stratum granulosum, which lies between the stratum spinosum, below, and the stratum lucidum, above, in stratified squamous keratinized thick skin of palms and soles. Thin skin, which covers the rest of the body, lacks a definite stratum lucidum and stratum granulosum.

Below the granulosum is the stratum spinosum, which is a multi-layered arrangement of cuboidal cells. Adjacent cells are joined by desmosomes, giving them a spiny appearance when the cells shrink during the staining process while the desmosomes hold firm. Their nuclei are often darkened (a condition called pyknosis), which is an early sign of cell death. Their fate is sealed because the nutrients and oxygen in interstitial fluid have become exhausted before the fluid is able to reach them by diffusion.

Stratum germinativum (also stratum basale or basal cell layer) is the layer of keratinocytes that lies at the base of the epidermis immediately above the dermis. It consists of a single layer of tall, simple columnar epithelial cells lying on a basement membrane. These cells undergo rapid cell division, mitosis, to replenish the regular loss of skin by shedding from the surface. About 25% of the cells are melanocytes, which produce melanin, which provides pigmentation for skin and hair.

The next lower strata of skin is the dermis, which consists of connective tissue and cushions the body from stress and strain. The dermis is tightly connected to the epidermis by a basement membrane. It also harbors many nerve endings that provide the sense of touch and heat. It contains the hair follicles, sweat glands, sebaceous glands, apocrine glands, lymphatic vessels and blood vessels. The blood vessels in the dermis provide nourishment and waste removal to its own cells, as well as the Stratum basale of the epidermis.

Beneath the dermis is the hypodermis, which is also called the subcutaneous layer, hypoderm, or superficial fascia. Its purpose is to attach the skin to underlying bone and muscle, as well as supplying it with blood vessels and nerves. It consists of loose connective tissue and elastin. The main cell types are fibroblasts, macrophages and adipocytes (the hypodermis contains 50% of body fat). Fat serves as padding and insulation for the body.

The term “parenterally administering” refers to modes of administration other than oral and topical administration.

The term “photosensitizer” or “photosensitizing agent” refers to an light-activatable compound that can be used in photodynamic therapy. The photosensitizers of the invention can produce a photochemical or phototoxic effect on a cell when light activated, i.e., produce a reactive species when light activated. The photosensitizers of the invention can include photosensitizer fragments and/or derivatives of known photosensitizers, which have the same or substantially the same function as the known photosensitizers, which means that function which is at least about 50% of the function of an original photosensitizer, more preferably about 60% or 70%, or still more preferably about 80% or 90%, or even more preferably about 95% or 99% the function of the known photosensitizer compound.

Photosensitizing agents can include, but are not limited to, chlorins, bacteriochlorins, phthalocyanines, porphyrins, purpurins, merocyanines, psoralens and pro-drugs (or pro-photosensitizers), such as aminolevulinic acid (ALA), which can be metabolized once administered to a subject to photosensitizing compounds, such as protoporphyrin. It is understood that the above photosensitizers, including those described elsewhere herein, are exemplary, and that other photosensitizers which are known or yet to be developed having the appropriate spectral characteristics may also be useful in the practice of the present invention. It will be understood further that “pro-photosensitizer,” e.g., 5-aminolevulinic acid, means any molecule, which when administered to a mammal is capable of being metabolized or otherwise converted to produce a photosensitizer, or is capable of stimulating the synthesis of an endogenous photosensitizer. It is contemplated that the pro-photosensitizer may be converted into a photosensitizer of interest or stimulate the synthesis of an endogenous photosensitizer at the site of the target disorder, e.g., a skin-related disorder. Alternatively, the pro-photosensitizer may be converted into a photosensitizer or stimulate the synthesis of an endogenous photosensitizer at a region remote from the skin lesion, after which the photosensitizer is transported to the skin lesion, for example, via the vasculature.

The term “subject” is used herein to refer to a living animal, including a mammal. The subject can be a human. The subject can have a skin disorder.

The term “obtaining,” as in “obtaining” the “ALA or derivative thereof,” is intended to include purchasing, synthesizing or otherwise acquiring the elements of the invention.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning described in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Photosensitizers

The present invention contemplates the use of any photosensitizer that may be used in accordance with the photodynamic therapy methods of the invention. The photosensitizers can be used both diagnostically or therapeutically, in accordance with the herein described methods. The photosensitizers of the invention can produce a photochemical or phototoxic effect on a cell when light activated, i.e., produce a reactive species when light activated. Any suitable photosensitizer or combination of photosensitizers can used. Photosensitizing agents can include, but are not limited to, chlorins, bacteriochlorins, phthalocyanines, porphyrins, purpurins, merocyanines, psoralens and pro-drugs (or pro-photosensitizers), such as aminolevulinic acid (ALA), which can be metabolized once administered to a subject to photosensitizing compounds, such as protoporphyrin. The photosensitizers of the invention can be amphiphilic, meaning that they share the opposing properties of being water-soluble, yet hydrophobic. The photosensitizers can be water-soluble in order to pass through the bloodstream systemically or be more readily delivered to tissues. In certain aspects, the photosensitizers can be hydrophobic in order to aid in their passage across cell membranes. Modifications, such as attaching polar residues (amino acids, sugars, and nucleosides) to the hydrophobic porphyrin ring, can alter polarity and partition coefficients to desired levels. Such methods of modification are well known in the art.

In specific aspects, photosensitizers of the present invention can absorb light at a relatively long wavelength, thereby absorbing light at low energy. Low-energy light can travel further or deeper through tissue, e.g., epidermis, dermis or subcutaneous hypodermis of skin, than high-energy light, i.e., has greater penetration potential. In one aspect, the photosensitizers of the invention can be excitable at wavelengths of light which are optimized for penetrating tissues, which can include light at wavelengths of between about 650 and about 800 nm.

The invention can also utilize photosensitzers that are derived from natural sources, such as porphyrins from red blood cells or chlorins found in chlorophyll. In some aspects, porphyrins found in red blood cells, which typically absorb at about 630 nm, can be modified to have optical spectra that have been “red-shifted”, in other words, absorbs lower energy light. Other naturally occurring compounds, such as chlorins found in chlorophyll (about 640 to about 670 nm) or bacteriochlorins found in photosynthetic bacteria (about 750 to about 820 nm), can be modified to have optical spectra that is red-shifted with respect to porphyrin.

In addition to those photosensitizers already listed above, the invention contemplates the use of porphyrins and/or hydroporphyrins as photosensitizers of the invention. Porphyrins and hydroporphyrins can include, but are not limited to, Photofrin® (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorin e₆ monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium(III) texaphyrin, or a functional derivative or fragment thereof, i.e., compounds that are chemically similar and/or are small portions of the original compound which perform the same or substantially the same function.

It will be appreciated that porphyrins, hydroporphyrins, benzoporphyrins, and derivatives thereof are all related in structure to hematoporphyrin, a molecule that is a biosynthetic precursor of heme, which is the primary constituent of hemoglobin, as well as certain enzymes. In certain aspects, the porphyrins of the invention can be excited at about 630 nm and may have an overall low fluorescent quantum yield and low efficiency in generating reactive oxygen species. Light at about 630 nm can only penetrate tissues to a depth of about 3 mm, however there are derivatives that have been ‘red-shifted’ to absorb at longer wavelengths, such as the benzoporphyrins BPD-MA (Verteporfin), which are also contemplated by the invention.

Porphyrin derivatives contemplated by the invention also include chlorins and bacteriochlorins, which can possess hydrogenated exo-pyrrole double bonds on the porphyrin ring backbone This feature can allow for absorption at wavelengths greater than about 650 nm. Chlorins are derived from chlorophyll, and modified chlorins, such as meta-tetrahydroxyphenylchlorin (mTHPC), have functional groups to increase solubility. Bacteriochlorins are derived from photosynthetic bacteria and are further red-shifted to about 740 nm.

Porphryin derivatives contemplated by the invention also can include purpurins, porphycenes, and verdins, which have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals. In a specific embodiment, the photosensitizer can be Antrin®, otherwise known as motexafin lutetium.

In particular, the methods and compositions of the invention utilize aminolevulinic acid (ALA) and derivatives thereof, e.g., 5-aminolevulinic acid, as the photosensitizer for treating and/or diagnosis of the target and/or skin-related disorders of the invention or derivatives thereof. ALA is a precursor in the heme biosynthetic pathway, and exogenous administration of this compound causes a shift in equilibrium of downstream reactions in the pathway. In other words, the formation of the immediate precursor to heme, protoporphyrin IX (PpIX), is dependent on the rate of aminolevulinic acid synthesis, governed in a negative-feedback manner by concentration of free heme. Conversion of protoporphyrin IX is slow, and where desired, administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm.

Without wishing to be bound by theory, all nucleated cells have at least a minimal capacity to synthesize protoporphyrin IX, since heme is necessary for the synthesis of various essential heme-containing enzymes. Certain types of cells and tissues can synthesize relatively large quantities of PpIX. Under normal conditions, the synthesis of PpIx in such tissues is under such tight feed-back control that the cells produce it at a rate just sufficient to match their need for heme. However, the usual rate-limiting step in the process, the synthesis of 5-aminolevulinic acid, can be bypassed by the provision of exogenous ALA, porphobilinogen, or other precursor of PpIX. Certain tissues and organs will then accumulate such a large excess of PpIX that they become both fluorescent and photosensitive. At least in the case of the skin, the PpIx appears to be synthesized in situ. It is known that oral and parenteral routes of ALA delivery leads to the induction of clinically useful concentrations of PpIX in certain benign and malignant tissues throughout the body, i.e., ALA exhibits preferential accumulation in rapidly growing cells. Only certain types of tissues synthesize and accumulate clinically useful amounts of PpIX when provided with an excess of ALA. By the expression “rapidly growing cell” is meant herein any lesion, abnormal cell or normal cell that exhibits cell growth substantially greater than that of the surrounding tissues and that preferentially accumulates protoporphyrin IX from exogenous ALA.

The present invention also contemplates the use of any suitable derivative of 5-aminolevulinic acid (ALA) for administration in accordance with the methods of the invention. ALA derivatives are known in the art and can include, for example, the ALA derivatives described in U.S. Pat. Nos. 7,335,684; 7,287,646; 7,247,655; 7,217,736; 6,992,107; 6,916,971; 6,897,238; 6,860,879; 6,710,066; 6,603,062; 6,583,317; 6,559,183; 6,492,420; 6,335,465; 6,034,267; 5,955,490; 5,907,058; 5,856,566; 5,520,905; 5,422,093; 5,344,974; 5,284,973; 5,234,940; and 5,079,262, each of which are incorporated herein by reference.

The present invention also contemplates use of “first-generation photosensitizers”, which are exemplified by the porphyrin derivative, Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a specific embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized (Richter et al., 1987; Aveline et al., 1994; Levy, 1994) and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically administered intravenously, with an optimal incubation time range from 1.5 to 6 hours. Verteporfin absorbs at 690 nm, and is activated with commonly available light sources. One tetrapyrrole-based photosensitizer having recent success in the clinic is MV0633 (Miravant).

The photosensitizers can have a chemical structure that includes multiple conjugated rings that allow for light absorption and photoactivation. Such specific compounds include motexafin lutetium (Antrin®) and chlorinₑ₆.

The photosensitizers of the present invention also include cyanines and other photoactive dyes. Cyanine and other dyes include but are not limited to a merocyanine, phthalocyanine, chloroaluminum phthalocyanine, sulfonated aluminum PC, ring-substituted cationic PC, sulfonated AlPc, disulfonated or tetrasulfonated derivative, sulfonated aluminum naphthalocyanine, naphthalocyanine, tetracyanoethylene adduct, crystal violet, azure β chloride, benzophenothiazinium, benzophenothiazinium chloride (EtNBS), phenothiazine derivative, phenothiaziniums such as rose Bengal, toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, or thionine.

Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maxima at about 680 nm. Phthalocyanines, belonging to a new generation of substances for PDT are chelated with a variety of diamagnetic metals, chiefly aluminum and zinc, which enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, where, for example, high tumor to tissue ratios (8:1) were observed after 1-3 hours. The cyanines are eliminated rapidly and almost no fluorescence can be seen in the tissue of interest after 24 hours.

Other photoactive dyes such as methylene blue and rose bengal, are also used for photodynamic therapy. Methylene blue is a phenothiazine cationic dye that is exemplified by its ability to specifically target mitochondrial membrane potential. Rose-bengal and fluorescein are xanthene dyes that are well documented in the art for use in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

In other aspects, the photosensitizers can be Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, and tetracyclines are other compounds that also exhibit photoactive properties and can be used advantageously in photodynamic therapy. Other photosensitizers that do not fall in either of the aforementioned categories have other uses besides photodynamic therapy, but are also photoactive. For example, anthracenediones, anthrapyrazoles, aminoanthraquinone compounds are often used as anticancer therapies (i.e., mitoxantrone, doxorubicin). Chalcogenapyrylium dyes such as cationic selena- and tellurapyrylium derivatives have also been found to exhibit photoactive properties in the range of about 600 to about 900 nm range, more preferably from about 775 to about 850 nm. In addition, antibiotics, such as tetracyclines and fluoroquinolone compounds, have demonstrated photoactive properties.

Any of the photosensitizers described herein can be prepared as pharmaceutically acceptable derivatives in the form of salts, esters, enol ethers, enol esters, acetals, ketals, hemiacetals, hemiketals, acids, bases, solvates, hydrates or prodrugs thereof using methods known in the art. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benz-imidazole, diethylamine and other alkylamines, piperazine and tris(hydroxy-methyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl ar heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

The photoreactive agents for use in the methods provided herein may be prepared from readily available starting materials by methods well known to those of skill in the art, or routine modification thereof, or are commercially available (e.g., from Sigma-Aldrich Chemical Co., Milwaukee, Wis.). Methods for preparation of the photoreactive agents are disclosed in U.S. patent application Ser. No. 09/078,329, filed May 13, 1998, entitled “Controlled Activation of Targeted Radionuclides”, Ser. No. 60/116,234, filed Jan. 15, 1999, entitled “Targeted Transcutaneous Cancer Therapy”, Ser. No. 09/271,575, filed Mar. 18, 1999, entitled “Targeted Transcutaneous Cancer Therapy”, Ser. No. 09/905,501, filed Jul. 13, 2001, entitled “Targeted Transcutaneous Cancer Therapy”, Ser. No. 09/905,777, filed Jul. 13, 2001, entitled “Non-invasive Vascular Therapy”, Ser. No. 60/175,689, filed on Jan. 12, 2000, entitled “Novel Treatment for Eye Disease”, Ser. No. 09/760,362, filed on Jan. 12, 2001, entitled “Novel Treatment for Eye Disease”, and Ser. No. 60/116,235, filed on Jan. 15, 1999, entitled “Non-invasive Vascular Therapy”, the disclosure of each of which is hereby incorporated by reference in its entirety. Methods for preparation of the photoreactive agents for use in the methods provided herein are also disclosed in, e.g., U.S. Pat. Nos. 6,319,273, RE37,180, 4,675,338, 4,693,885, 4,656,186, 5,066,274, 6,042,603, 5,913,884, 4,997,639, 5,298,018, 5,308,861, 5,368,841, 5,952,366, 5,430,051, 5,567,409, 5,942,534, and U.S. patent application Publication No. 2001/0,022,970. Methods for the preparation of taporfin sodium, also known as mono-L-aspartyl chlorin e6 are disclosed in, e.g., U.S. Pat. Nos. RE37,180, 4,675,338 and 4,693,885.

The photosensitizers of the invention can also be attached to a targeting moiety which targets it to a specific cell or tissue structure, e.g., ligands for adipose tissue.

The photosensitizing agents of the invention also can be conjugated to specific ligands reactive with a target, such as receptor-specific ligands or immunoglobulins or immunospecific portions of immunoglobulins, permitting them to be more concentrated in a desired target cell or tissue of interest. The photosensitizing agents may be further conjugated to a ligand-receptor binding pair, which includes, but are not limited to: biotin-streptavidin; and antigen-antibody. This conjugation may permit lowering of the required dose level since the material is more selectively target and less is wasted in distribution into other tissues whose destruction must be avoided. In aspects relating to targeting and reducing adipose tissue, the invention further contemplates targeting moieties that are suitable for directing the photosensitizers of the invention to adipose tissue, e.g., anti-adipocyte antibodies or antibodies that are specific for an adipocyte-associated protein or extracellular component or receptor.

The targeting moieties of the invention can bind to a defined population of cells, e.g., cells of the skin or subcomponent thereof. In various aspects, they can bind a receptor, an oligonucleotide, an enzymatic substrate, an antigenic determinant, or other binding site present on or in the target cell population. Accordingly, the targeting moiety can be a molecule or a macromolecular structure that targets specific cells.

Desirable characteristics for the targeting moieties can include: specificity for one or more cells or tissues of the targeted treatment site, affinity and avidity for such sites, and stability with respect to conditions of coupling reactions and the physiology of the organ or tissue of use. Specificity need not be narrowly defined, e.g., it may be desirable for a targeting molecule to have affinity for a broad range of target cells. The targeting moiety, when incorporated into a composition of the invention, should be nontoxic to the cells of the subject.

Targeting moieties can be selected from the sequences of naturally occurring proteins and peptides, from variants of these peptides, and from biologically or chemically synthesized peptides. Naturally occurring peptides which have affinity for one or more target cells or tissues can provide sequences from which additional peptides with desired properties, e.g., increased affinity or specificity, can be synthesized individually or as members of a library of related peptides. Such peptides can be selected on the basis of affinity for the target cell or tissue.

Targeting moieties need not be limited to peptide compositions, but can be lectins, polysaccharides, steroids, and metalloorganic compositions, and other known materials. Targeting moieties can be comprised of compositions that are composed both of amino acids and sugars, such as mucopolysaccharides. A useful targeting moiety can be partially lipid and partially peptide in nature, such as low density lipoprotein. Serum lipoproteins especially high density and low density lipoproteins (HDL and LDL) can bind to bacterial surface proteins (Emancipator, K. et al., Infect. Immun. 60:596-601, 1992). HDL, and especially reconstituted HDL, neutralizes bacterial lipopolysaccharide both in vitro and in vivo (Wurfel M M et al., J. Exp. Med. 181:1743-1754, 1995). Endogenous LDL can protect against the lethal effects of endotoxin and Gram negative infection (Netea, M., et al., J. Clin. Invest. 97:1366-1372, 1996). The appropriate binding features of the lipoproteins to bacterial surface components can be identified by methods of molecular biology known in the art, and the binding feature of lipoproteins can be used as the targeting moiety in photosensitizer compositions of the present invention.

Molecules, e.g., peptides, other than antibodies and members of a high affinity ligand pairs, can be used to target a photosensitizer composition according to the invention to a target cell or tissue. Targeting moieties can be modified or refined. Once an example of a targeting moiety of reasonable affinity has been provided, one skilled in the art can alter the disclosed structure (of a polylysine polypeptide, for example), by producing fragments or analogs, and testing the newly produced structures for modification of affinity or specificity. Examples of methods which allow the production and testing of fragments and analogs are discussed in U.S. Pat. No. 6,462,070.

### Administered Formulations

The photosenstizer compositions of the invention can be used therapeutically photodynamic therapy of a skin-related disorder and other target disorders. It is considered that the choice of the appropriate photosensitizer or pro-photosensitizer, formulation, dosage, and mode of administration will vary depending upon several factors including, for example, the skin-related disorder (or other disorder) to be treated, the bodily location of the treatment site, whether the treatment site is an external or internal bodily site, and the age, sex, weight, and size of the mammal to be treated, and may be varied or adjusted according to choice. The photosensitizer or pro-photosensitizer is administered so as to permit an effective amount of photosensitizer to be present in the target region. As used herein, the term “effective amount” means an amount of photosensitizer suitable for photodynamic therapy, i.e., the photosensitizer is present in an amount sufficient to produce a desired photodynamic reaction at the target site. The photosensitizer or pro-photosensitizer may be administered in a single dose or multiple doses over a period of time to permit an effective amount of photosensitizer to accumulate in the target region. Fluorescence spectroscopy or other optical detection or imaging techniques may be used to determine whether and how much photosensitizer is present in the target region.

The compositions comprising the photosensitizer or pro-photosensitizer of the invention may be formulated in conventional manner with one or more physiologically acceptable carriers or excipients, according to techniques well known in the art, as described in more detail below. Compositions may be administered systemically, transdermally, intratracheally, or parentarally.

In one particular aspect, the photosensitizer or pro-photosensitizer compositions of the invention can be provided in a form adapted for parenteral administration, for example, by intramuscular, intradermal, subcutaneous, intraperitoneal, or intravenous injection. Alternative pharmaceutical forms thus include plain or coated tablets, capsules, suspensions, and solutions containing the active component optionally together with one or more inert conventional carriers and/or diluents, for example, with corn starch, lactose, sucrose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearylalcohol, carboxymethylcellulose or fatty substances such as hard fat or suitable mixtures thereof.

When the photosensitizers of the invention are formulated as an injection, in general, the carrier may desirably be physiological saline, various buffered solutions, aqueous solutions of a sugar such as glucose, inositol, mannitol and the like, or a glycol such as ethylene glycol, polyethylene glycol and the like. Further, the inventive compositions may also be formulated into a lyophilised preparation in association with an excipient which may be a sugar such as inositol, mannitol, glucose, mannose, maltose, sucrose and the like, or an amino acid such as phenylalanine and the like. Upon administration, the lyophilised preparation may be dissolved into a solvent suitable for the injection, for example, a liquid available for intravenous injection, which may be sterile water, physiological saline, aqueous solution of glucose, solution of electrolytes and aqueous solution of amino acids, and the like.

The dosage of the photosensitizer composition of the invention to be used as the active ingredient, or its salt or ester can depend on the age, body weight and symptoms of patients, and the purposes of the therapeutic treatment, and other factors. The dosage is to give an effective amount of the photosensitizer composition to accumulate in the tissues or cells sought to be treated. The photosensitizer composition may be administered continuously or intermittently as long as a total dosage of the photosensitizer composition does not exceed a specific level which is decided in view of the results of animal tests and various circumstances.

When administered parenterally, the total dosage of the photosensitizer composition of this invention can be administered with appropriate adjustments being done in view of the way of administration, the conditions of patients such as age, body weight and sex, as well as foods and medicines concurrently administered. Suitable dosage and administration frequency of the photosensitizer composition of this invention under given conditions can be determined by expert physician through the tests of determining optimal dosage in light of the above-mentioned guidelines. These guidelines for administration also apply to the photosensitizer compositions of the invention.

The compositions of the invention can be delivered in various formulations including, but not limited to, liposome, peptide-bound, polymer-bound, or detergent-containing formulations. Those of ordinary skill in the art are well able to generate and administer such formulations. The composition can be soluble under physiological conditions, in aqueous solvents containing appropriate carriers or excipients, or in other systems, such as liposomes, that may be used to administer the conjugate to a subject.

Photosensitizer compositions that are somewhat insoluble in an aqueous solvent can be applied in a liposome, or a time release fashion, such that illumination can be applied intermittently using a regimen of periods of illumination alternating with periods of non-illumination. Other regimens contemplated are continuous periods of lower level illumination, for which a time-release formulation is suitable.

Compositions of the present invention can be administered in a therapeutically effective amount by a variety of methods known in the art. In one aspect, a photosensitizer composition of the invention may be administered parenterally. The phrase “administered parenterally” as used herein means modes of administration other than oral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. As used herein, a “therapeutically effective amount” refers to that amount of a photosensitizer composition that, when administered to a subject, is sufficient to decrease a symptom of a skin-related disorder of interest.

As will be appreciated by the skilled artisan, the route and/or mode of administration will vary depending upon the desired results. A photosensitizer composition according to the invention can be contained in a pharmaceutically acceptable excipient or carrier. Included, without limitation, are any and all solvents, dispersion media, coatings, antibacterial and antifungal agents, isotonic and absorption delaying agents, and the like that are physiologically compatible, or other active ingredients, such as pain-relieving medicaments (e.g. antinociceptive agent). The use of such media and agents for pharmaceutically active substances is well known in the art. Advantageously, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

In one aspect, the carrier may protect the compound against rapid release, for example, a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In another aspect, the photosensitizer compositions can be administered by combination therapy, i.e., combined with other agents. For example, the combination therapy can include a composition of the present invention with at least one other photosensitizer, at least one antibiotic, or other conventional therapy.

In a particular aspect, the photosensitizers of the invention can be co-administered with an antinociceptive agent. As used herein, the term “antinociceptive agent” refers to a compound or drug that are used to alleviate pain in a subject. Antinociceptive agents can include, for example, any pain-relieving compound, including, steroids, analgesics, barbiturates or opioids, and derivatives thereof. The term “co-administration,” as used herein, refers to the administration of a photosensitizing agent of the invention substantially at the same time as an antinociceptive agent. Co-administration can include administering the photosensitizers and the antinociceptive agents sequentially or at the same time (e.g., an admixture of both or simultaneous administration of separate compositions) to the same treatment site or systemically. In one aspect, co-administration can be by way of a transdermal patch, or equivalent device, whereby the patch or equivalent device contains one or both of the co-administered compounds.

Antinociceptive agents are well known in the art. In one embodiment, the antinociceptive agent is one which is non-systemic, but instead, is administered and is effective through local application to a treatment site.

Antinociceptive agents can include opioids, which include, but are not limited to, compounds based on or derived from morphine-like compounds and analogs. The opioid can be, but is not limited to, ethylmorphine, hydromorphine, morphine, oxymorphone, codeine, levorphanol, oxycodone, pentazocine, propoxyphene, fentanyl, sufentanil, lofentanil, morphine-6-glucuronide, buprenorphine, methadone, etorphine, butorphanol, nalorphine, nalbuphine, naloxone benzoylhydrazone, bremazocine, ethylketocyclazocine, U50,488, U69,593, spiradoline, naltrindole, [D-Pen²,D-Pen⁵]enkephalin (DPDPE), [D-Ala²,Glu⁴]deltorphin, and [D-Ser²,Leu⁵]enkephalin-Thr⁶ (DSLET), [D-Ala²,MePhe⁴,Gly (ol)⁵] enkephalin, and β-endorphin, dynorphin A, dynorphin B and small molecule and combinatorial chemistry products thereof. As new opioids are discovered they can be effectively used in accordance with the present invention.

Antinociceptive agents that also can be used with the invention can include lidocaine and other local anesthetics, including, but not limited to, bupivacaine, mepivacaine, ropivacaine, tetracaine, etidocaine, chloroprocaine, prilocalne, procaine, benzocaine, dibucaine, dyclonine hydrochloride, pramoxine hydrochloride, benzocaine, and proparacaine.

The co-administration methods of the invention can also involve the administration of more than one antinociceptive agent.

Additional antinociceptive agents that can be used in accordance with the invention are known in the art, for example, in U.S. Pat. Nos. 4,608,376, 4,803,208, 6,509,028, 6,790,855, and 6,825,203, as well as in U.S. Published Application No. 2002/0004484, 2002/0013331, 2002/0052319, 2002/0192288, and 2003/0124190, each of which are incorporated herein by reference in their entireties.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation.

In a particular aspect, the present invention provides for dose-dependent administration of the photosensitizers of the invention such that targeted and selective delivery of a photosensitizer is directed to a specific component or structure of the targeted skin. The invention relates, in part, to the discovery that certain photosensitive agents, when administered intradermally at particular depths, are selectively targeted to different microscopic dermatological structures or sites depending on the concentration or amount administered. Because singlet oxygen and radical intermediates generated during PDT are highly reactive and cause oxidative damage precisely at the sites where they are generated, the methods and compositions of the invention provide an easy and effective clinical approach for targeting therapy and/or diagnosis of a specific tissue site or structure. More in particular, the present invention contemplates that different drug concentrations determine and affect different skin targets. It has been found that, advantageously, that high doses of injected photosensitive agents, in combination with high-fluence red light, can cause potent vascular PDT reactions. It has also been found that moderate doses of injected photosenstive agents can selectively target eccrine glands of the skin. Further, it was found that administration of very low doses of certain photosensitive agents selectively targeted fat and deep vessels without clinical inflammation. Thus, injected ALA-PDT can be used for treatment of various skin-related disorders, such as, vascular lesions, eccrine disorders, or subcutaneous fat removal in accordance with the invention.

As used herein, the term “high doses” or a high dose of a photosensitive agent of the invention refers to administering a solution comprising about 0.25% to about 1.00% or more w/v of the photosenstive agent of the invention. In accordance with the invention, use of high dose formulations of the photosensitizers of the invention cause potent vascular PDT-related damage.

As used herein, the term “moderate dose” or a moderate dose of a photosensitive agent of the invention refers to administering a solution comprising about 0.03% to about 0.5%, and more preferably about 0.06% to about 0.2% w/v of the photosenstive agent of the invention, and still more preferably about 0.06% of the photosenstive agent of the invention. In accordance with the invention, use of moderate dose formulations of the photosensitizers of the invention selectively cause eccrine gland PDT-related damage.

As used herein, the term “low dose” or a low dose of a photosensitive agent of the invention refers to administering a solution comprising less than about 0.03%, more preferably less than about 0.02%, still more preferably less than about 0.016% to about 0.0005% w/v of the photosenstive agent of the invention. In accordance with the invention, use of low dose formulations of the photosensitizers of the invention selectively cause adipose PDT-related damage.

In one particular aspect, the invention provides methods and compositions utilizing the pro-photosensitizer, ALA. As described above, ALA is a precursor in the synthesis of PpIX, a naturally occurring photosensitizer, which itself is a precursor in the synthesis of heme. See, e.g., U.S. Pat. Nos. 5,079,262, 5,211,938, and 5,955,490. Both ALA and PpIX are naturally present in the body and, therefore, in general, induce few or no side-effects. It is believed that all nucleated cells have at least a minimal capacity to synthesize PpIX. Typically, the synthesis of PpIX is regulated so that it is produced in cells at a rate just sufficient to satisfy the need for heme. Although the synthesis of ALA is a rate-limiting step in the synthesis of heme, it is believed that this step can be bypassed by providing exogenous ALA, or other precursors of PpIX.

In aspects relating to the treatment and/or diagnosis of skin-related disorders or reduction of adipose tissue, the ALA of the invention can be administered by intradermal injection of a solution containing the ALA to a suitable depth in the skin. The ALA-containing solution or formulation, which can be prepared in accordance with the invention described herein elsewhere, can be prepared and administered in a dose-dependent manner to effectuate a specific or targeted administration of the photosensitizer to a target tissue or component of the skin. In one aspect, the ALA solution is formulated at a low-dose level, comprising less than about 0.03%, more preferably less than about 0.02%, still more preferably between about 0.016% to about 0.0005% w/v of the photosenstive agent of the invention. When administered by intradermal injection, low dose formulations of ALA are specifically targeted to subcutaneous adipose tissue, and thus, ALA can be utilized in methods of photodynamic therapy to reduce subcutaneous adipose tissue in accordance with the invention.

In another aspect, the ALA solution is formulated at a moderate-dose level, comprising less than about 0.03%, more preferably less than about 0.02%, still more preferably between about 0.016% to about 0.0005% w/v of the photosenstive agent of the invention. In accordance with the invention, use of low dose formulations of ALA selectively causes adipose PDT-related damage.

In yet another aspect, the ALA solution is formulated at a moderate-dose level, comprising about 0.03% to about 0.5%, and more preferably about 0.06% to about 0.2%, and still more preferably about 0.06% w/v of the ALA. The use of moderate dose formulations of the ALA selectively causes eccrine gland PDT-related damage.

In still another aspect, the ALA solution is formulated at a high-dose level, comprising about 0.25% to about 1.00% or more w/v of ALA. In accordance with the invention, use of high dose formulations of ALA causes potent vascular PDT-related damage.

In methods relating treating and/or diagnosis of skin-related disorders, the photosensitizers of the invention, e.g., ALA, can be administered by intradermal injection. The depth of the infection can be adjusted or determine by the skilled artisan and can depend on the particular skin-related disorder desired to be treated. In one aspect, the photosensitizers are injection into the skin at a depth of less than about 4 mm, or more preferably less than about 3 mm or even less than about 2 mm, or still more preferably about between 2-3 mm. However, the skilled artisan can inject the photosensitizers of the invention at any suitable depth as seen fit such that the photosensitizers are optimally injected proximate or close to the desired site of treatment. For example, in aspects relating to the targeted reduction of adipose tissue, the photosenstizers of the invention, e.g., ALA, is injected at a depth sufficient to reach the subcutaneous adipose layer.

In addition, it will be appreciated that the photosensitizer or pro-photosensitizer dosage can be adjusted with respect to the irradiation parameters, including, for example, wavelength, fluence, fluence rate, irradiance, duration of the light, and the time interval between administration of the photosensitizer or pro-photosensitizer and the irradiation, and the cooling parameters, if surface cooling is desired. All of these parameters can be adjusted to produce a photodynamic reaction resulting from activation of the photosensitizer in the target region that is effective with minimal side effects.

Appropriately selected radiation that is capable of penetrating the skin and activating the photosensitizer present in the region of irradiation is desired. The skilled artisan will be able to select a light source having the appropriate spectral properties based on knowledge in the art. It will be appreciated that the penetration of light is a function of its spectral properties. For example, estimated penetration depths as a function of wavelength are as follows: 440 nm will penetrate to about 0.6 mm of skin, 460 nm will penetrate to about 1.0 mm, 480 nm will penetrate to about 1.3 mm, 500 nm will penetrate to about 1.6 mm, 520 nm will penetrate to about 1.7 mm, 580 nm will penetrate to about 1.4 mm, 600 nm will penetrate to about 4.2 mm, 620 nm will penetrate to about 5.2 mm, 640 nm will penetrate to about 5.8 nm, and 660 nm will penetrate to about 6.3 mm. The preceding estimates are not meant to restrict the invention in any way and are only meant as a guide. The relationship between choice of suitable spectral properties of the light source, and the depth of the site of treatment, will be well known to the skilled artisan and can readily be determined. For example, the method of Jacques (Jacques, 1992, Proc. Spie. 1645: 155-165) can be used to estimate the penetration depths of light sources having different spectral characteristics.

Any suitable device or equipment is contemplated by the present invention to deliver or administer the photosensitizers of the invention. The type or mode of delivery can depend upon a number of factors, including the bodily location of the desired treatment site (e.g., internal bodily site, intravascular site, external bodily site, such as, skin), the type of target disorder sought to be treated (e.g., skin-related disorder or adipose reduction), the extent of the disorder, and the type of bodily tissue into which administration shall be made. For example, for skin-related disorder, needles or equivalent devices can be used to deliver the photosensitizers of the invention. For internal bodily sites, such as targeted inner adipose tissue, catheter-related devices can be utilized to deliver the photosensitizers to internal sites using minimally invasive procedures.

In one aspect, the photosensitizers of the invention can be applied to affected areas of the skin with the aid of a transdermal skin patch. Skin patches are well known in the art. Such methods can also include any suitable delivery carrier which would assist in the penetration of the photosensitizers of the invention. Such penetrants are well known in the art. The transdermal patches of the invention may advantageously be configured with devices suitable for injecting or directly delivering the photosensitizers of the invention to the dermis or subcutaneous region of the treated skin, e.g., sharp projections or needles that pass through the epidermis to deliver the compounds of the invention to regions of interest of the skin.

The iterations delineated above are not intended as limiting with respect to the nature of the conjugate photosensitizer compositions of the invention, or to a particular route of the administration.

### Photoactivation/Illumination

The photosensitizer compositions of the invention are photoactivated in both therapeutic and diagnostic uses according to the invention. For therapeutic uses, administration of a photosensitizer composition according to the invention is typically followed by a sufficient period of time to allow accumulation thereof at the target site. For example, the ALA of the invention, once delivered, needs sufficient time to be metabolized by the body to protoporphyrin IX. Upon encountering the target tissue to be treated or evaluated diagnostically, the photosensitizers can, subsequently, be activated by irradiation. This is accomplished by applying light of a suitable wavelength and intensity, for an effective length of time, at the site of the infection for therapeutic uses, or at the site of reaction for diagnostic uses. As used herein, “irradiation” refers to the use of light to induced a chemical reaction of a photosensitizer.

Photoactivating dosages depend on various factors, including the amount of the photosensitizer administered, the wavelength of the photoactivating light, the intensity of the photoactivating light, and the duration of illumination by the photoactivating light, and the depth at which the photosensitizer exists in the target tissue. Thus, the dose can be adjusted to a therapeutically effective dose or to a dose suitable for diagnostics by adjusting one or more of these factors. Such adjustments are within the level of ordinary skill in the art.

Irradiation of the appropriate wavelength for a given compound may be administered by a variety of methods. Methods for irradiation include, but are not limited to, the administration of laser, nonlaser, or broad band light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments that provide transillumination. With therapeutic embodiments, delivery of the light to a recessed, or otherwise inaccessible physiological location can be facilitated by flexible fiber optics (implicit in this statement is the idea that one can irradiate either a broad field, such as the lung or a lobe of the lung, or a narrow field where bacterial cells may have localized).

The suitable wavelength, or range of wavelengths, will depend on the particular photosensitizer(s) used and/or depth of the photosensitizer in a target tissue (e.g. skin), and can range from about 350 nm to about 550 nm, from about 550 nm to about 650 nm, from about 650 nm to about 750 nm, from about 750 nm to about 850 nm and from about 850 nm to about 950 nm.

In some aspects, target tissues are illuminated with red light. Given that red and/or near infrared light best penetrates mammalian tissues, photosensitizers with strong absorbances in the range of about 600 nm to about 900 nm can be suitable for activation of administered photosensitizers of the invention. For photoactivation, the wavelength of light is matched to the electronic absorption spectrum of the photosensitizer so that the photosensitizer absorbs photons and the desired photochemistry can occur. Wavelength specificity for photoactivation generally depends on the molecular structure of the photosensitizer. Photoactivation can also occur with sub-ablative light doses. Determination of suitable wavelength, light intensity, and duration of illumination is within ordinary skill in the art.

In embodiments utilizing aminolevulinic acid or derivatives thereof, the invention may be carried out using any suitable wavelength of light that is sufficient to excite the ALA to produce a phototoxic effect. The spectral characteristics of ALA are well known. ALA is known to have an excitation peak at about 450 nm, which falls within the blue light range of about 400 nm to about 450 nm. ALA exhibits another peak at about 509 nm, which falls within the green light spectrum of about 500 nm to about 510 nm. ALA further exhibits a peak within the yellow light range of about 540 nm to about 585 nm. In addition, ALA exhibits a peak of about 635 nm, which is within the red light range of about 630 nm to about 635 nm.

Thus, in certain aspects the invention provides that any of the above wavelengths can be used to irradiate the ALA of the invention or its derivatives. In particular, where ALA is delivered to an internal bodily sites, i.e., excluding the skin, any of the above wavelengths of excitatory light can be used to illuminate the ALA compositions of the invention since any number of light-delivery devices (e.g., fiber optics) can be used to directly illuminate a treatment site. Administration of PDT light to ALA administered in the skin preferably utilizes red light to take advantage of penetration potential of red light. In one embodiment, the red light can be between about 620 and about 645 nm.

The fluence and irradiance of the administered light can be adjusted by conventional means. In one embodiment, fluence of the red light is between about 100 and about 300 J/cm² and the irradiance of the red light is between about 30 and about 200 mW/cm².

With therapeutic uses, the effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the photosensitizer can alter these values. In general, photosensitizers with longer absorbing wavelengths and higher molar absorption coefficients at these wavelengths are more effective photodynamic agents.

The light for photoactivation can be produced and delivered to the treatment side or to a diagnostic reaction by any suitable means known in the art. Photoactivating light can be delivered from a light source, such as a laser or optical fiber. Optical fiber devices that directly illuminate the treatment site or a diagnostic reaction can deliver the photoactivating light. For example, for therapeutic uses, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Optical fibers can also be passed through arthroscopes. In addition, light can be transmitted by percutaneous instrumentation using optical fibers or cannulated waveguides. For open surgical sites, suitable light sources include broadband conventional light sources, broad arrays of light-emitting diodes (LEDs), and defocused laser beams.

Delivery can be by all methods known in the art, including transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

In aspects where treatment is desired, the dosage of photosensitizer composition, and light activating the photosensitizer composition, can be administered in an amount sufficient to produce a phototoxic species. For example, where the photosensitizer is chlorineₑ₆, administration to humans is in a dosage range of about 0.1 to about 10 mg/kg, preferably about 1 to about 5 mg/kg more preferably about 2 to about 4 mg/kg and the light delivery time is spaced in intervals of about 30 minutes to about 3 days, preferably about 12 hours to about 48 hours, and more preferably about 24 hours. The light dose administered is in the range of about 2-500 J/cm², preferably about 5 to about 50 J/cm², and more preferably about 5 to about 10 J/cm². The fluence rate is in the range of about 20 to about 500 mw/cm², preferably about 50 to about 300 mw/cm² and more preferably about 100 to about 200 mw/cm². There is a reciprocal relationship between photosensitizer compositions and light dose, thus, determination of suitable wavelength, light intensity, and duration of illumination is within ordinary skill in the art.

Dosage regimens can be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Irradiation of the appropriate wavelength for a given compound for the therapeutic or diagnostic methods of the invention may be administered by a variety of wavelengths. Methods for irradiation include, but are not limited to, the administration of laser, nonlaser, or broad band light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments that provide transillumination.

The wavelength and power of light can be adjusted according to standard methods known in the art to control the production of phototoxic species or a fluorescence response. Thus, under certain conditions (e.g., low power, low fluence rate, shorter wavelength of light or some combination thereof), a fluorescent species is primarily produced from the photosensitizer and any reactive species produced has a negligible effect. These conditions are easily adapted to bring about the production of a phototoxic species. For example, where the photosensitizer is chlorineₑ₆, the light dose administered to produce a fluorescent species and an insubstantial reactive species is less than about 10 J/cm, preferably less than about 5 J/cm and more preferably less than about 1 J/cm. Determination of suitable wavelength, light intensity, and duration of illumination for any photosensitizer is within the level of ordinary skill in the art.

A sample can be illuminated with a wavelength of light selected to give a detectable optical response, and observed with a means for detecting the optical response. Equipment that is useful for illuminating the present compounds and compositions of the invention includes, but is not limited to, hand-held ultraviolet lamps, mercury arc lamps, xenon lamps, lasers and laser diodes, as well as LED (light emitting diode) lights. These illumination sources are optically integrated into laser scanners, fluorescence microplate readers or standard or microfluorometers, or any other suitable known means for detecting and/or measuring the signal (e.g., a fluorescence signal).

The herein disclosed photosensitizers may, at any time after or during an assay, be illuminated with a wavelength of light that results in a detectable optical response, and observed with a means for detecting and measuring the optical response. Upon illumination, such as by an ultraviolet or visible wavelength emission lamp, an arc lamp, a laser, or even sunlight or ordinary room light, the fluorescent compounds, including those bound to the complementary specific binding pair member, display intense visible absorption as well as fluorescence emission. Selected equipment that is useful for illuminating the fluorescent compounds of the invention includes, but is not limited to, hand-held ultraviolet lamps, mercury arc lamps, xenon lamps, argon lasers, laser diodes, and YAG lasers. These illumination sources can be optionally integrated into laser scanners, fluorescence microplate readers, standard or mini fluorometers, or chromatographic detectors. Any suitable computer software for measuring, processing and displaying images and/or data pertaining to the process of detecting and measuring signal sequences will be known to the skilled artisan and are contemplated by the invention.

Fluorescence emissions can be optionally detected by visual inspection, or by use of any of the following devices: CCD cameras, video cameras, photographic film, laser scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, a fluorescence microscope or a fluorometer, the instrument is optionally used to distinguish and discriminate between the fluorescent compounds of the invention and a second fluorophore with detectably different optical properties, typically by distinguishing the fluorescence response of the fluorescent compounds of the invention from that of the second fluorophore. Where a sample is examined using a flow cytometer, examination of the sample optionally includes isolation of particles within the sample based on the fluorescence response by using a sorting device.

The above description is not meant to limit the means, methods or instrumentation that can be used to detect, measure, quantitate, and analyze signals (e.g., fluorescence) produced by the photosensitizers of the invention in connection with those embodiments pertaining to the diagnostic methods of the invention. Any suitable means, methods or instrumentation for detecting, measuring, quantitating and analyzing photosensitizer signals are contemplated.

### Methods of Treatment

It is contemplated that the methods and compositions of the invention are useful in the treatment and/or diagnosis of any number of tissue disorders, including, but not limited to skin disorders affecting or relating to the skin or any skin component, such as a cell or tissue of the dermis or subcutaneous layers, including any vascular cell or structure, nerve cell or structure, muscle cell or structure, adipose cell or structure, or gland, hair or nail component that is associated with or forms a part of the skin. Skin-related disorders can include any neoplastic and non-neoplastic dermatological conditions. In certain aspects, the skin-related disorders include vascular malformations or lesions, including, for example, warts, hemangiomas, Port wine stains, Sturge-weber Syndrome, congenital vascular malformations, secondary neovascularizations (including those associated with cancers), macular degeneration and other similar malformations. In certain other aspects, the skin-related disorders of the invention include skin gland disorders, such as eccrine or appocrine gland disorders, including, for example, hyperhidrosis, hidradenitis, suppurativa, syringomas, bromohydrosis, or any benign or malignant tumor related to such glands. In still other aspects, the skin-related disorders that are treatable by the methods and compositions of the invention include dermatological adipose, e.g., adipose tissue associated with the subcutaneous layer of the skin.

As used herein, the term “target disorder” encompasses skin-related disorders as defined herein, but also extends to any bodily site-internal or external-comprising a target or diseased tissue of interest.

The present invention further contemplates the treatment of basal cell, baso-squamous and squamous cell carcinomas and other lesions of the skin, mucosa (respiratory, digestive, and vaginal), endometrium and urothelium. Treatment sites—which could include lesions or cellular abnormalities—generally are those of epithelial or endothelial origin, including, but not limited to, those lesions or abnormalities involving (i) skin, circulatory system and conjunctiva; (ii) the lining of the mouth, pharynx, esophagus, stomach, intestines and intestinal appendages, rectum, and anal canal; (iii) the lining of the nasal passages, nasal sinuses, nasopharynx, trachea, bronchi, and bronchioles; (iv) the lining of the ureters, urinary bladder, and urethra; (v) the lining of the vagina, uterine cervix, and uterus; (vi) the parietal and visceral pleura; (vii) the lining of the peritoneal and pelvic cavities, and the surface of the organs contained within those cavities; (viii) the dura mater and meninges; (ix) any tissues or suspensions of body fluids containing abnormal cells, including blood, that can be made accessible to photoactivating light either in vitro, at time of surgery, in vivo through the skin via surface irradiation or via an optical fibre inserted through a needle; (x) all exocrine glands and associated ducts, including: mammary glands, sebaceous glands, ceruminous glands, sweat glands, and lacrimal glands; mucus-secreting glands of the digestive, urogenital, and respiratory systems; salivary glands; liver, bile ducts, and gall bladder; pancreas (exocrine component); gastric and intestinal glands; prostate; Cowper's, Bartholin's and similar glands. It is also contemplated that cell abnormalities in the gonads (testes and ovaries), thymus, spleen, lymph nodes, bone marrow, lymph and blood would also be treated according to the invention. Tumors of the nervous system or connective tissues (sarcomas) can also be treated according to the present invention.

The term, target disorder, also encompasses unwanted adipose tissue occurring anywhere in the body, including adipose tissue associated with organs, bodily cavities, abdomen, or even endovascular fat.

The present invention, in certain aspects, also contemplates that certain advantageous accompanying procedures can be performed depending on the target disorder or skin-related disorder desired to be treated and/or diagnosed. It is contemplated, that the efficacy of the treatment may be improved by descaling the diseased tissue prior to irradiation. For example, in the case of psoriasis, psoriatic scales may be removed by chemical and/or physical processes well known in the art. During a physical process, the psoriatic scales may be removed, for example, by dermabrasion. Alternatively, the psoriatic scales may be removed chemically by application of a descaling agent, for example, salicylic acid. This can be accomplished by applying a 5% salicylic acid preparation to the region of interest on a daily basis for a period of one week before irradiation. Other chemical treatments include, for example, the topical application of a drug, for example, a topical corticosteroid to the site of the lesion.

Under certain circumstances of the invention, it may be advantageous to minimize thermal injury to the bodily area under treatment by the methods of the invention. This can be accomplished by cooling the skin surface prior to, contemporaneous with, and/or after irradiation. Cooling can be facilitated by one or more cooling systems known and used in the art. Cooling systems useful in the practice of the invention may include, without limitation: blowing a cold stream of gas, for example, cold air, cold nitrogen or cold helium, onto the surface of the treatment area; spraying a cold liquid stream onto the surface of the treatment area; conductive cooling using a cold contact surface which does not interfere with the irradiation, for example, a cooled transparent optical material, such as a cooled sapphire tip (e.g., U.S. Pat. No. 5,810,801); applying a low boiling point, non-toxic liquid, for example, tetrafluoroethane or chlorodifluoromethane, onto the surface of the treatment area, to cool the tissue surface by evaporative cooling, or applying a low boiling point non-toxic liquid onto the surface of the target tissue combined with blowing a stream of gas in the vicinity of the liquid to remove at least a portion of the liquid (e.g., U.S. Patent Application No. 20010009997A1).

Cooling can also be facilitated by a dynamic cooling device (DCD), such as a DCD manufactured by Candela Corp. (Wayland, Mass.). Applications of the DCD have been described in the art (e.g., U.S. Pat. No. 5,820,626). The DCD provides a timed spray of fluid onto the surface of the skin, prior to, contemporaneous with, and/or after irradiation. Unlike steady-state cooling, for example, an ice cube held against the tissue, dynamic cooling primarily reduces the temperature of the most superficial layers of the skin. For example, it has been estimated that the use of tetrafluoroethane as a cryogen may result in a drop in surface-temperature of about 30-40° C. in about 5-100 milliseconds.

In some aspects, the light delivery system can include an integrated cooling system for cooling the skin surface prior to, contemporaneous with, and/or after irradiation. Accordingly, such a light delivery system would be multi-functional, i.e., capable of both delivering a beam of irradiation and cooling the surface of the skin at the same time. By way of example, an integrated hand piece can be used to apply a beam of light from a laser source and a cryogen spray to a preselected region of the treatment area. Application of the light (heat energy) together with surface cooling can be used to limit thermal injury to the specific portions of the treatment area, e.g., the skin, for example, where the capillaries of the superficial horizontal plexus, are present in a psoriatic plaque, while preserving the epidermis.

The light delivery and cooling systems can also comprise separate systems. The cooling system may comprise a container of a cold fluid. Cooling the surface of the skin can be accomplished by applying the cold fluid onto the skin which then extracts heat from the skin on contact. In such an embodiment, a light delivery system comprises, for example, a hand piece containing optics for directing, collimating or focusing the irradiation beam onto the targeting region of the skin surface. The light beam can be carried from the energy source, for example, a laser, to the hand piece by, for example, an optically transparent fiber, for example, an optical fiber. Coolant from a separate reservoir can be applied to the surface of the targeted region. In this embodiment, coolant from the reservoir flows to a dispensing unit separate from the energy delivery system via tubing connecting the reservoir and the dispensing unit. The coolant, once dispensed, can be retained in situ on the surface of the targeted region by a ring, for example, a transparent ring, which can be attached to the energy delivery system.

### Articles of Manufacture

The present invention also provides that the photoreactive agents or compositions comprising same may be packaged as articles of manufacture containing packaging material, a photoreactive agent or pharmaceutically acceptable derivative thereof, which is effective for photodynamic therapy or diagnosis, within the packaging material, and a label that indicates that the photoreactive agent, or pharmaceutically acceptable derivative thereof, is used for photodynamic therapy or diagnosis, and instructions for use in accordance with the methods and claims disclosed herein. The package can also contain any suitable device for administering the compositions of the invention, including a syringe or transdermal patch or the like.

The articles of manufacture can further include one or more active ingredient that may be suitably used for co-administration in accordance with the invention. In one aspect, the active ingredient can be an antinociceptive agent, such as an opioid, analgesic, anesthetic, and the like, such as, but not limited to lidocaine and other local anesthetics, including, but not limited to, bupivacaine, mepivacaine, ropivacaine, tetracaine, etidocaine, chloroprocaine, prilocalne, procaine, benzocaine, dibucaine, dyclonine hydrochloride, pramoxine hydrochloride, benzocaine, and proparacaine. The antinociceptive agent can also include opioids, which include, but are not limited to, ethylmorphine, hydromorphine, morphine, oxymorphone, codeine, levorphanol, oxycodone, pentazocine, propoxyphene, fentanyl, sufentanil, lofentanil, morphine-6-glucuronide, buprenorphine, methadone, etorphine, butorphanol, nalorphine, nalbuphine, naloxone benzoylhydrazone, bremazocine, ethylketocyclazocine, U50,488, U69,593, spiradoline, naltrindole, [D-Pen²,D-Pen⁵]enkephalin (DPDPE), [D-Ala²,Glu⁴]deltorphin, and [D-Ser²,Leu⁵]enkephalin-Thr⁶ (DSLET), [D-Ala²,MePhe⁴,Gly (ol)⁵] enkephalin, and β-endorphin, dynorphin A, dynorphin B and derivatives, small molecule, combinatorial chemistry products thereof.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the photoreactive agents provided herein are contemplated as are a variety of treatments for any disease or disorder in which photodynamic therapy or diagnosis is indicated.

Having now generally described the invention, the same will be more readily understood through reference to the following examples, which are provided by way of illustration, and are not intended to be limiting of the present invention.

### EXAMPLES

The invention will now be further described by way of the following non-limiting examples. It should be appreciated that the invention should not be construed to be limited to the examples that are described; rather, the invention should be construed to include any and all applications provided herein and all equivalent variations within the skill of the ordinary artisan.

### Example 1

Photodynamic therapy (PDT) consists of activating a photosensitizer that targets specific pathways, cells or structures. Aminolevulinic acid (ALA), the first committed precursor of heme synthesis, is used as a topical agent for PDT following the first description by Kennedy and colleagues (Kennedy et al. 1990). Metabolism of ALA leads to accumulation of porphyrins, notably the potent photosensitizer protoporphyrin IX (PpIX). In the presence of oxygen and light, PpIX transfers energy to generate singlet oxygen (¹O₂), a potent oxidizing agent. Topical ALA-PDT is known in the art. ALA-PDT has also been used as a cosmetic for the rejuvenation of photodamaged skin.

The distribution of tissue structures affected by PDT is determined by a combination of photosensitizer, light and oxygen distributions. Most of the light sources used for ALA-PDT emit blue or red light related to absorption bands of PpIX. For the common treatment of actinic keratoses, blue light and topical ALA are appropriate because the target lesions are very superficial. Although blue light at ˜415 nm is about 20 times more strongly absorbed by PpIX than red light at ˜635 nm, penetration of blue light in vivo is limited to less than about one millimeter (Anderson and Parrish 1981). In contrast, red light is deeply penetrating, and the irradiance can be increased to compensate for the lower absorption of red light. The maximum depth of topical ALA-PDT treatment using red light is therefore limited mainly by drug penetration. This limitation is important for basal cell carcinoma and other deep targets.

To enhance the penetration of topical ALA solutions, a variety of different approaches have been proposed, including delivery by injection. In this Example, the inventors have proposed intradermal ALA administration followed by red light activation for targeting deeper cutaneous structures. However, basic information about intradermal ALA administration is lacking. The minimum photosensitizing ALA concentration has not been determined, biological distribution of PpIX has not been well documented after intradermal injection of ALA, and the distribution of tissue damage caused by intradermal ALA-PDT at various doses is unknown. This Examples describes PpIX distribution according to fluorescence microscopy, and ALA-PDT effects when different concentrations of injected ALA are activated by 635 nm light in an animal model.

### Methods and Materials

### Animals

Three female domestic Yorkshire swine weighing 30 to 50 kg were used in the study, which was approved by the MGH Institutional Subcommittee for Research Animal Care. Porcine and human skin have similar histology and physiology, with notable differences. Pigs have smaller sebaceous glands and a thicker dermis. However their thick stratum corneum, lack of fur and metabolism of topical or injected ALA are similar to humans (Meyer 1996), (de Blois, Thissen et al. 2001).

Animals were anesthetized with intramuscular (IM) Telazol/Xylazine (4.4 mg/kg and 2.2 mg/kg) and inhaled Isoflurane 2.0% with 3.0 L/min in oxygen after fasting overnight. Before the photodynamic therapy (PDT) experimental procedure, buprenorphine HCL (Buprenex®, 0.005-0.0010 mg/kg) was injected IM. After recovery from anesthesia, half of this dose was given to reduce pain. The next morning (approximately 24 hours after the procedure), half of the initial dose was again injected (IM) and then the animal was again anesthetized prior to biopsies. Temperature-controlled blankets were used under the animals to maintain core temperature while under anesthesia. Euthanasia was performed with pentobarbital 100 mg/kg IV while under anesthesia.

### ALA Solutions

5-Aminolevulinic acid hydrochloride from Biosynth International Inc. (Naperville, Ill., USA) was diluted in normal saline (0.9% Sodium Chloride, Hospira, Inc., Illinois, USA) into different concentrations. ALA solutions were titrated in halves from 1.000% to 0.0005%. Osmolarity was not corrected. ALA solution pH varied with concentration from acidic to neutral (1.000%≈pH 1; 0.500%≈pH 3; 0.250 to 0.008%≈pH 5; 0.004 to 0.0005%≈pH 7). Normal saline was used as a control (pH=7). Aqueous solutions of ALA are unstable over long periods depending on the pH and the concentration of the solution [(Novo et al. 1996), (Elfsson et al. 1999), (Bunke et al. 2000)]. In this study, ALA solutions were not buffered because buffering can induce a delay in PpIX formation (Bech et al. 1997). Unbuffered solutions were prepared immediately before intradermal injection. A volume of 0.05-cc/cm² skin area was injected at a depth of 2 to 4 mm, using a special 4 mm-long 30 gauge needle (TSK STEiJECT, Toshigi-ken, Japan). The beveled outlet of this needle covers the distal 1 mm near its tip.

For each concentration, 12 repetitions were performed for statistical validation. One centimeter separated each site of application from others at the same concentration. After ALA administration, the skin was occluded with plastic sheeting (Saran wrap) and covered with aluminum foil, to avoid light exposure during a 3-hour period for ALA metabolism.

### Study Design

Each animal's flanks were cleansed, clipped of hair, cleansed and rinsed again, dried with a towel, mapped and divided into 4 cm×7 cm rectangular areas for the test spots. Each test area received an injection of a given ALA concentration, with appropriate control sites, which received either no treatments, ALA alone, or light exposure alone. After the ALA injections, the skin surface was wiped clean again. Three hours after ALA administration, each test area was used for repeated measurements of (1) digital fluorescence photography to document the gross distribution of porphyrin synthesis; and (2) 8-mm skin biopsies obtained from at least 2 sites for each condition prior to light exposure, for evaluation of porphyrin distribution by fluorescence microscopy. After the 3-hour-incubation skin biopsies, red light exposures were performed using a 635 nm LED array (Omnilux, Photo Therapeutics, Inc, Cheshire, UK), delivered at 100 mW/cm² irradiance. Biopsies were taken from the control sites at the same times as from test sites, to control for the unlikely possibility that the small amount of ALA injected would cause a systemic level sufficient to stimulate remote synthesis of porphyrins. Measurements and biopsies were repeated after 24 hours, prior to euthanasia.

### Fluorescence Photography

Fluorescence photography was performed using a 420 nm bandwidth excitation filter, and >600 nm long pass emission filter mounted on a digital camera (Nikon D70, Nikon Corporation, Tokyo, Japan) with fixed-focus and fixed-field with 10 cm field of view. A fluorescent tape (Small Flags—Pink, Redi-Tag Corporation, Cypress, Calif., USA) was attached to the fixed-field area to compare the fluorescence produced from the skin. Fluorescence photography was performed at baseline, after 3 hours of incubation, immediately after red light exposure, and 24 hours after the application of the drug for each ALA application and control test sites.

### Clinical Examination of Inflammatory Response to PDT

Clinical examination and grading of cutaneous responses were performed before, during and 1 hour after the end of the last red light exposure. This is sufficient time for an acute inflammatory response to develop. The following day, before euthanasia, final clinical evaluation was performed. Inflammatory response was graded from 0 to 3 for each site, where 0 represents no reaction; 1, mild (any redness or edema); 2, moderate (prominent redness and/or edema) and 3, severe reaction (signs of necrosis, blistering, purpura).

### Fluorescence Microscopy and Histopathology Examination

From each experimental skin area, a series 8 mm punch biopsies were taken and immediately split vertically in half with a razor blade. For each biopsy, half was prepared as a fresh-frozen specimen to localize the PpIX accumulation by fluorescence microscopy. The other half was processed for routine hematoxylin-eosin (H&E) staining. At baseline (B0), after 3 hours of incubation (B1), and after 24 hours (B2) a biopsy was performed in each area. Biopsies for fluorescence microscopy were also taken from sites receiving light exposure, just after the light was administered. Frozen sections were cut into 15 μm thick samples and observed using fluorescence microscopy Zeiss Axiophot, Carls Zeiss MicroImaging GmbH, Gottinger, Germany at 10× magnification; samples were also imaged by confocal microscopy (True Confocal Scanner—Leica TCSNT, Leica Mikroscopie und Systeme GmbH, Heidelberg, Germany) at 40× magnification. Routine H&E histology was performed to analyze PDT effect within the skin structures. Biopsy sites were separated from each other by at least 1 cm. Results from H&E slides were analyzed by a blinded dermatopathologist who rated inflammatory responses in the epidermis, dermis, hair follicles, sebaceous glands, eccrine and appocrine glands, fat and muscle after PDT on a scale from 0 to 3, where 0 represents no reaction; 1, mild reaction; 2, moderate reaction; 3, severe reaction (associated with necrosis and apoptosis).

### Results

### Digital Fluorescence Gross Photography Analysis

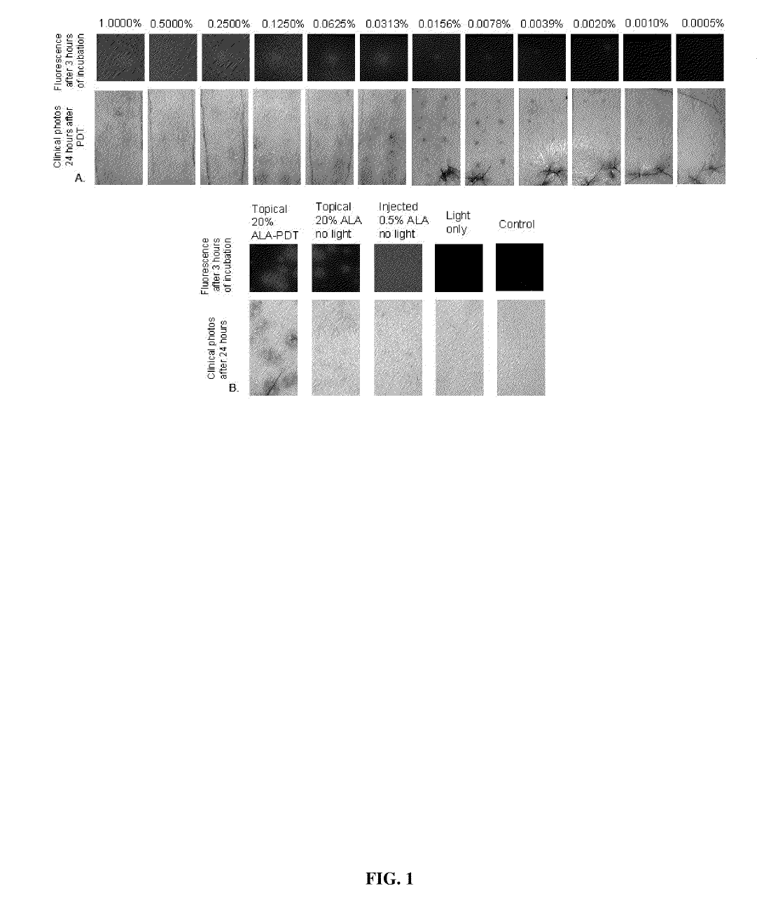

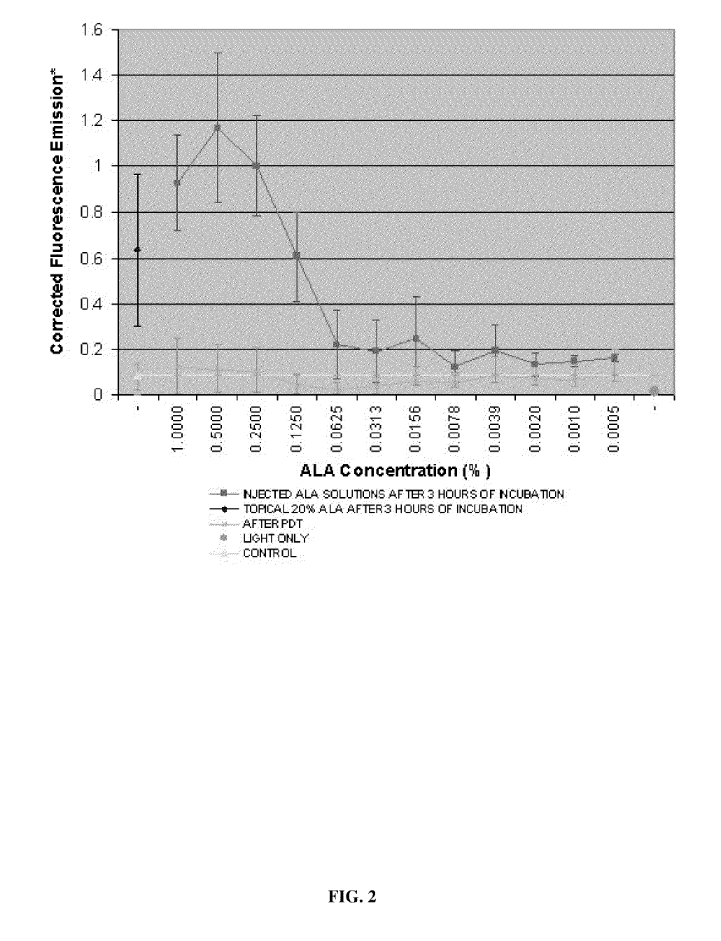

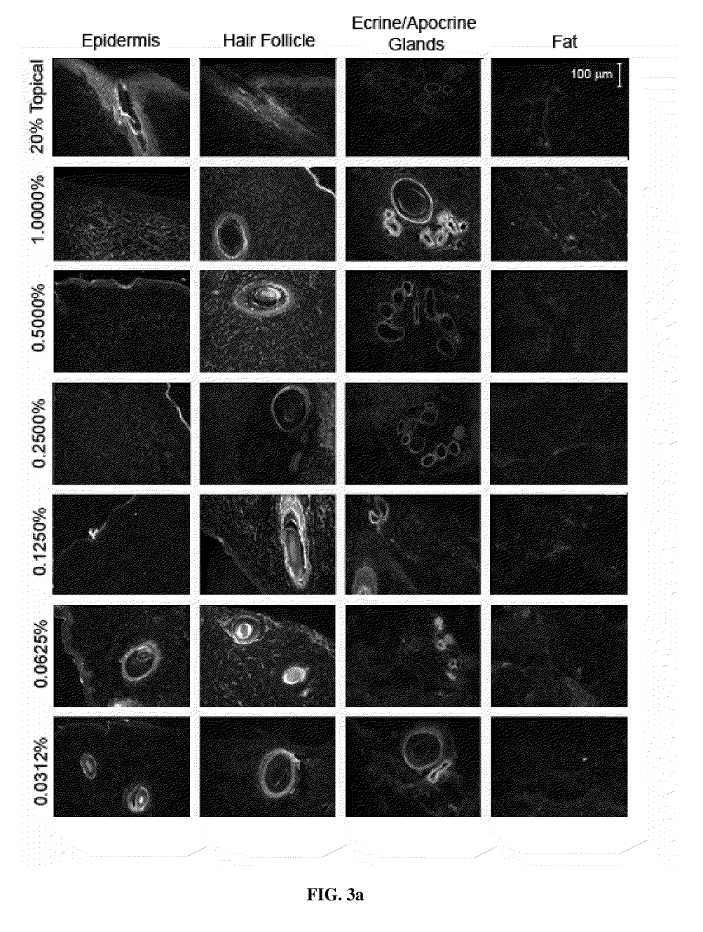

Porphyrin accumulation represented by positive fluorescence photographs was observed 3 hours after intradermal administration of 1.0% down to 0.004% ALA diluted solutions (see FIG. 1). Immediately after red light PDT, all sites showed no fluorescence, corresponding to complete photobleaching of porphyrins. Every gross fluorescent photograph was analyzed using the WCIF—Image J 1.37c software—Wayne Rasband National Institutes of Health, USA. In this software, pixel analysis is transformed into numeric values. Based on the fluorescence produced from a fluorescence standard within each image, pixel values were corrected to minimize errors resulting from slight differences in photographic flash exposures. Statistical analysis was performed using Microsoft® Excel 2000 and Microcal™ Origin software. After this pixel value analysis, fluorescence was statistically significantly increased compared with the uninjected control sites, down to an injected ALA concentration of 0.004% (p=). For ALA concentrations less than or equal to 0.002%, there were no statistically significant variation in fluorescence when ALA-injected sites were compared to each other (p=0.137). However, these low dose ALA-injected sites still showed significant fluorescence when compared to “control” and to “light only” (p≦2×10⁻⁵). Based on this data, 2-4 mm deep injected ALA concentrations lower than 0.0005% produced a low but detectable level of porphyrin accumulation, while injected ALA concentrations greater than 0.002% produced dose-dependent porphyrin accumulation that was readily detected by the gross fluorescence method. Endogenous porphyrins are also present in tissue without stimulation by ALA. The light-only control sites showed significantly lower fluorescence when compared to unexposed control sites (p<0.001), which is consistent with photobleaching of natural porphyrins present in the skin. This data is summarized and plotted in FIG. 2.

### Fluorescence Microscopy Analysis

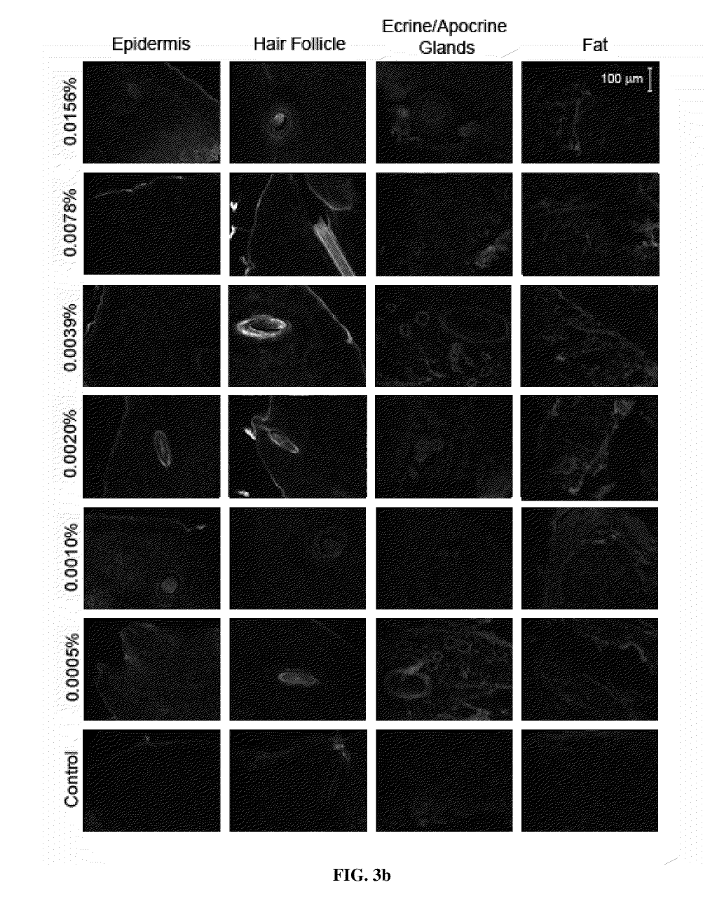

Fluorescence microscopy from fresh frozen sections prior to PDT light activation revealed porphyrin accumulation of various intensities in different cutaneous structures: epidermis, hair follicles, sebaceous glands, eccrine glands and fat (FIGS. 3a and 3b). Despite variations due to section thickness, size of structures, and photobleaching caused by the microscopy excitation source, porphyrin fluorescence was clearly higher after injection of more concentrated ALA solutions, and was negative at the control sites. Full thickness epidermal fluorescence was observed only after injection of the higher ALA concentrations (1.0% and 0.5%). At ALA concentrations below 0.06%, epidermal fluorescence was negative but there was positive fluorescence in the stratum corneum. Sebaceous glands showed fluorescence down to and including 0.50% ALA concentration. Fluorescence of superficial and deep hair follicles varied, and was mostly present from 1% to 0.03% ALA, while at lower concentrations only the hair shafts showed positive fluorescence, which was consistent with autofluorescence when compared to control sites. Statistical analysis of porphyrin fluorescence in the sebaceous glands was difficult due to their reduced number and size in young porcine skin. In contrast, eccrine glands are numerous, and there was strong porphyrin fluorescence after ALA injections of ≧0.03% concentration, and less intense but still positive fluorescence down to 0.005% ALA, even when adjacent hair follicles did not show any fluorescence. Some vessels also fluoresced after injection of 0.50% ALA concentration. Superficial fat showed strong fluorescence after ALA injection at all concentrations tested, fading with dilution. Subcutaneous fat fluorescence was present even after injection of very low concentration ALA (0.0005%). Porphyrin fluorescence in fat was mainly septal.

### Clinical Evaluation

ALA injections caused a mild transient “bleb” papule that quickly flattened, leaving behind only a small erythematous dot at the site of the needle puncture. This minimal reaction did not change over the 3-hour period of incubation prior to light exposures. During red light irradiation, areas with higher ALA concentrations (from 0.016% to 1.0%) developed intense vasospasm, seen clinically as pallor. These large areas of skin became uniformly cold compared to the immediately adjacent untreated areas. Areas near the experimental biopsies became purpuric after PDT exposure. The degree of responses (pallor/purpura) was directly proportional to ALA concentration. Concentrations from 0.25%-1.00% ALA produced extensive purpura with skin necrosis generally occurring after 24 hours. Nikolski sign was negative in all sites. Evaluations after 24 hours are shown in FIG. 1. Light exposure 3 hours after injection of ALA concentrations less or equal to 0.016%, produced only erythematous macules near each injection puncture site, while the lowest concentration (0.0005%) left no significant clinical residuum after light exposure.

Examination of sites receiving topical 20% ALA and light exposure after 24 hours produced a non-uniform skin response, with much greater inflammation around hair follicles. In contrast, injected ALA produced a much more uniform gross skin reaction, with the degree of inflammation directly proportional to the ALA concentration (FIG. 1).

### Histopathology Evaluation

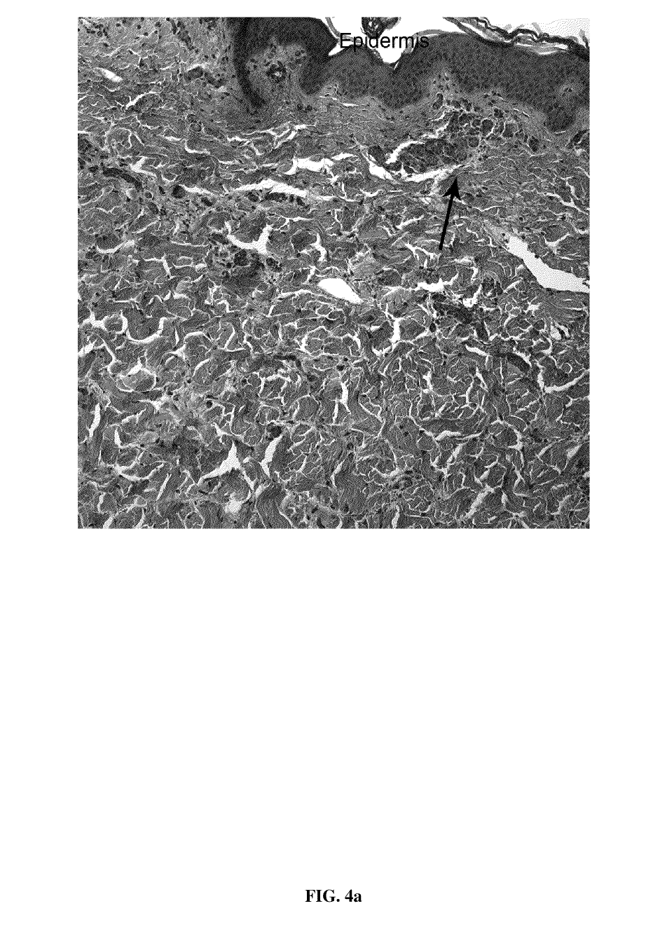

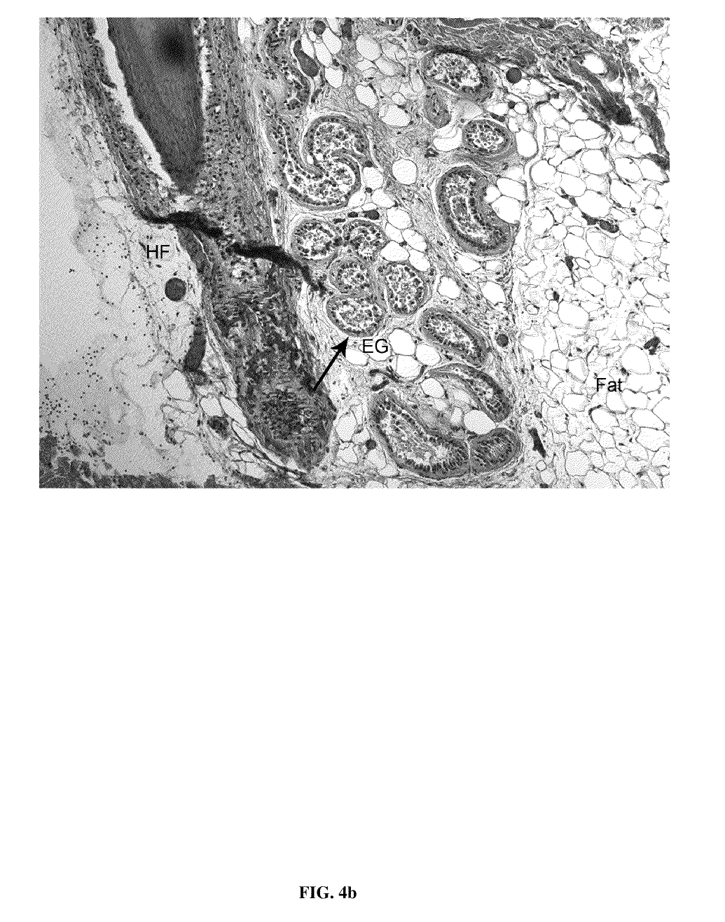

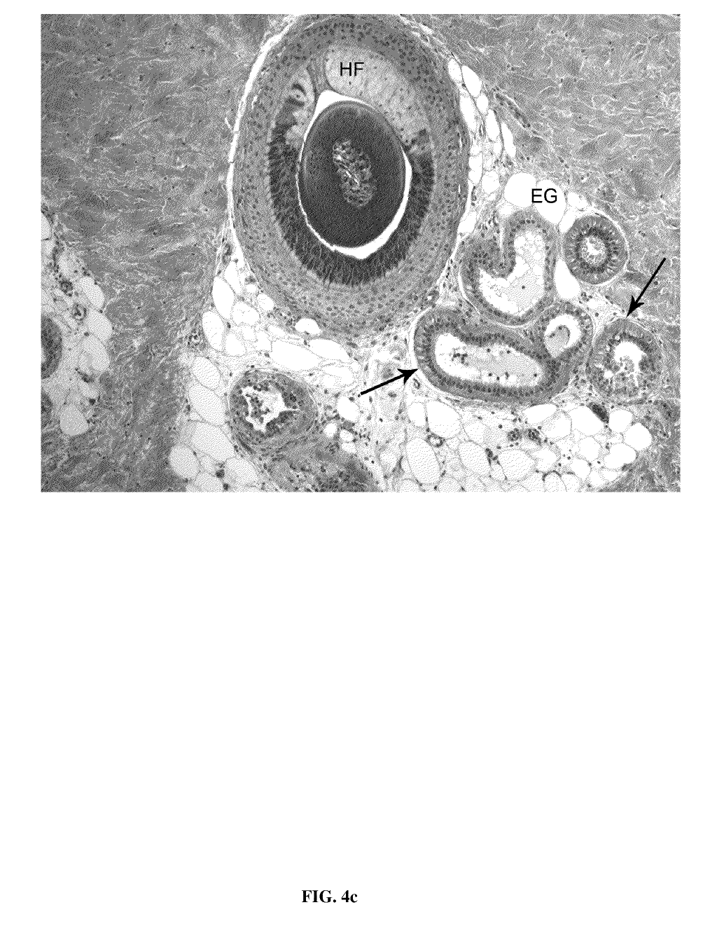

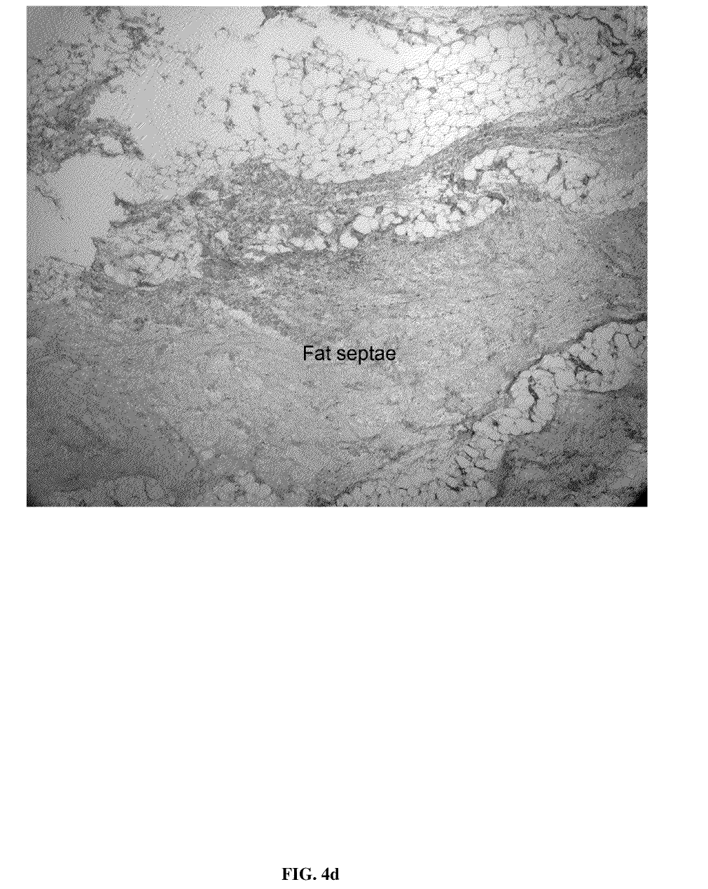

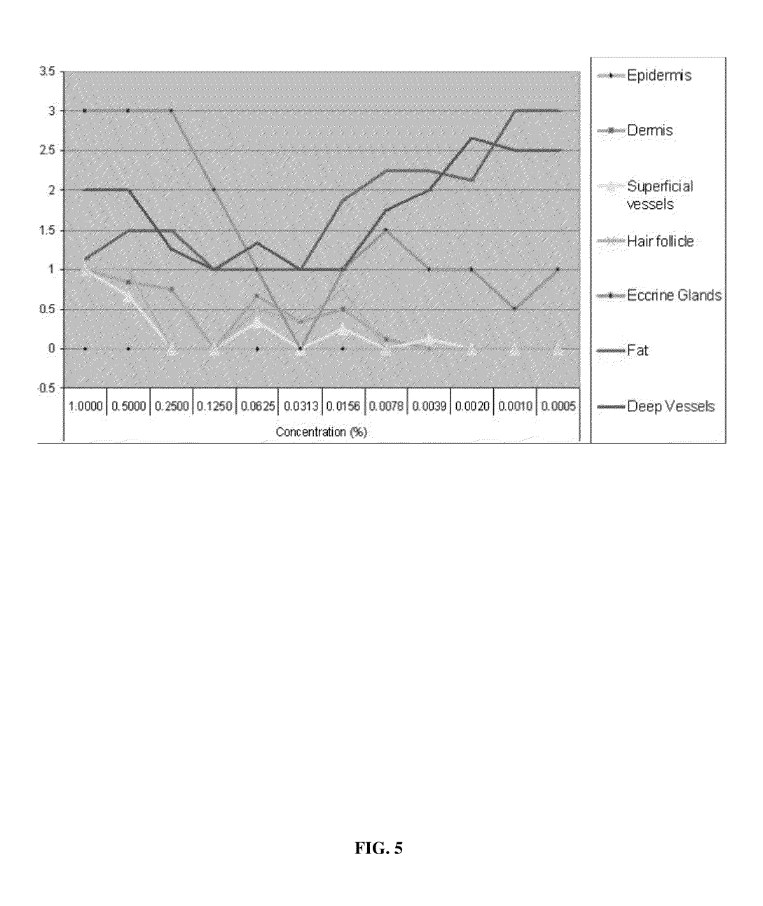

Biopsies taken 24 hours after PDT exhibited varying degrees of inflammation that were proportional to the concentration of ALA, with direct correlation to the clinical observations. The epidermis in all samples showed little or no reaction (FIG. 4a). Solutions from 0.25-1.00% showed extensive perivascular and vascular mixed inflammatory cell infiltrates, predominantly composed of mixed lymphocytes and eosinophils in the superficial and deep vessels associated with thrombosis, vessel necrosis and erythrocyte extravasation (FIG. 4a, 4b). The severity of these findings in superficial vessels decreased progressively with decreasing ALA concentrations from 1% to 0.015%. Surprisingly, in deep vessels of the fat septae, an inverse trend with ALA concentration was observed, with maximal vascular destruction occurring after light exposure of sites injected with the most diluted ALA solutions (0.0005%). In all cases, both arteries and veins were affected. Septal vascular destruction after low-concentration ALA PDT was also associated with fat necrosis (FIG. 4d). Fat damage was observed with all concentrations, but it was more intense with the more diluted ALA solutions (0.0005-0.016%). Furthermore, mild perifollicular inflammation was also observed mainly with higher drug concentrations (1%-0.016%). Massive eccrine gland necrosis with presence of neutrophils and eosinophils was observed (FIG. 4a, 4c) after light exposure in all ALA concentrations, but more so with concentrations varying from 1% to 0.06%. With 0.06% ALA, selective destruction of some eccrine glands was observed, with little or no inflammation in the epidermis, dermis or other skin structures. However, superficial fat also showed mild inflammation. The inflammation in different skin structures produced from varying ALA concentrations is summarized in FIG. 5.

### Discussion

The data indicated that intradermally injected ALA solutions target different microscopic tissue sites depending on the concentration of injected ALA, and furthermore, that these sites are largely different from those targeted by topical ALA-PDT. Singlet oxygen and radical intermediates generated during PDT are highly reactive, causing oxidative damage precisely at the sites where they are generated. Therefore, it is the microscopic distribution of a photosensitizer and/or oxygen within the tissue that largely determines “targeting” of different tissue structures. If our findings translate to human skin, it may be possible to achieve substantially different clinical effects simply by adjusting injected ALA concentration, and the depth of injection. Deeply penetrating red light, at a high fluence and at a fluence rate capable of both activating and fully photobleaching all porphyrins present throughout the skin was intentionally used in the example. Under these conditions, the sites of tissue injury are expected to correspond to the porphyrin and oxygen microscopic distribution, not the light distribution. For any given tissue site capable of synthesizing porphyrins from injected ALA, one also would expect greater photodynamic effect with higher ALA concentrations. Although this was generally true, surprisingly fat was more intensely and more selectively damaged after administration of the lowest concentration ALA injected solution. Furthermore, the data show that tissue responses after intradermal ALA are quite different from those after topical ALA, which is more commonly used in clinical practice.

Indications for topical ALA-PDT are limited in part by penetration of the drug and light into the skin. The specific uptake of topical, injected or iontophoresed ALA into specific skin structures, is unknown. It strongly appears that a direct route of topical ALA uptake occurs through “pores”, i.e. the infundibulum and perhaps deeper portions of the pilosebaceous unit. This is a likely explanation for the rapid appearance of porphyrin fluorescence in hair follicles and sebaceous glands after topical administration. It is well established that 635 nm light penetrates deeply into skin (Anderson and Parrish 1981). This is the deepest-penetrating wavelength light capable of activating porphyrins. The data show that red light affects epidermal, dermal, and subcutaneous target structures after injection of ALA into the region of deep dermis. When treatment of deeper targets is the goal, as in nodular skin cancer, topical ALA-PDT is not efficient (Martin, Tope et al. 1995; Rhodes, Tsoukas et al. 1997; Svanberg et al. 1994). In contrast, injected ALA certainly delivers the photosensitizer directly to the dermis and superficial fat. In this study, it was observed that injections of even low concentrations of ALA solution could induce porphyrin accumulation, down to and including 0.0005%. With lower ALA concentrations ≦0.002%, porphyrin fluorescence was limited to the area where the drug was injected (2-4 mm deep), similar to observations of Thiessen et al. (Thissen, de Blois et al. 2002). Localization of low-concentration ALA at the depth of injection may explain why porphyrin fluorescence was localized only in superficial fat at these concentrations.

Injected ALA-PDT clearly targets different skin structures than topical ALA-PDT, and the target sites are ALA concentration-dependent. While topical application creates porphyrin accumulation and PDT reactions mainly in epidermis and hair follicles, injected ALA creates relatively less reaction in these epithelial skin structures. Eccrine glands and subcutaneous fat showed strong porphyrin fluorescence, even after injection of low ALA concentrations. Epithelial skin structures—hair follicles, sebaceous glands and epidermis, showed fluorescence only after injection of ALA concentrations ≧0.25%. A trivial explanation for this, could be that a small amount of the injected ALA solution leaked from the injection tract to the skin surface, mimicking topical ALA-induced porphyrin distribution by a topical application route. However, the injected ALA concentrations were all low compared with 20% topical ALA concentration used for comparison. Epithelial porphyrins after ALA injection are more likely due to diffusion of the injected ALA through the dermis and basement membranes underlying the epithelial structures.

It is believed that eccrine-targeted PDT is a novel observation for ALA or any other photosensitizer. Selective eccrine gland damage was observed when a moderate ALA concentration was used (˜0.06%), suggesting that injected ALA-PDT might be useful for treatment of eccrine gland disorders such as syringomas and hyperhidrosis. The eccrine selectivity of injected ALA-PDT might be explained by peripheral benzodiazepine receptor expression (Morgan et al. 2004). In our study, apocrine glands also showed signs of some preferential damage, suggesting potential use of injected ALA-PDT for hidradenitis suppurativa and similar disorders.

In addition, high ALA doses (≧0.25%) in combination with high fluence red light caused a potent vascular PDT reaction. Vascular damage was an impressive and unexpected finding in this study, in both superficial and deep vessels. Superficial vessels were damaged more with PDT using higher ALA concentrations, whereas deeper vessels were paradoxically damaged more by PDT using low ALA concentrations. Both arteries and veins were affected. It is well known that intravenous or parenteral PDT drugs tend to cause vascular damage, for at least two reasons. First, vessels tend to be targeted because of oxygen availability (Henderson and Fingar 1987). Another factor is PDT drug distribution to vessels via intravascular delivery and often, lipoprotein receptor-mediated uptake. ALA is not lipophilic, and in our study did not arrive to the tissue by a vascular route. Furthermore, the study did not reveal much porphyrin synthesis in vessels by fluorescence microscopy. It is concluded that oxygen distribution is the most likely reason for the vascular damage by intradermal ALA-PDT. This study suggests that appropriate doses of intradermal ALA might be useful for treatment of vascular proliferations and/or malformations, e.g. hemangiomas or port wine stains.

It has also been observed that very low concentrations (0.016 to 0.0005%) of ALA injected 2-4 mm deep can cause PDT-induced localized fat necrosis without clinical evidence of inflammation. Fat necrosis was associated with septal vascular damage, and followed the same paradoxical dependence on ALA concentration as the deep vascular damage, with the lowest ALA concentrations causing the greatest damage. The reason for this paradoxical dependence is unknown. Deep dermis and upper fat are at the same depth at which ALA solution was injected in this study. Injected solutions of ALA tend not to migrate to other areas (de Blois, Thissen et al. 2001), such that at low concentrations the amount of ALA reaching other skin sites was probably insufficient to cause a PDT reaction beyond the local injection site. However, that would not per se explain why higher concentrations of injected ALA should lead to less damage of the deep vessels and fat. A possible explanation is related to the relatively poor blood supply of fat. At higher ALA concentrations, oxygen depletion during PDT exposure may also deplete oxygen supply to fat, reducing fat injury.

In conclusion, injected ALA-PDT is a novel therapeutic modality. By this study, it is observed that different drug concentrations determine and affect different skin targets. High doses of injected ALA solutions (≧0.25%) in combination with high-fluence red light caused a potent vascular PDT reaction. Moderate dose injected ALA (˜0.06%), selectively targeted eccrine glands. Very low doses (≦0.016%) targeted fat and deep vessels without clinical inflammation. Potentially, injected ALA-PDT could be used for treatment of vascular lesions, eccrine disorders, or subcutaneous fat removal.

### REFERENCES

Ammann R, Hunziker T and Braathen L R. (1995). Topical photodynamic therapy in verrucae. A pilot study. Dermatology 191(4):346-347.
Anderson R R and Parrish J A. (1981). The optics of human skin. J Invest Dermatol 77(1):13-19.
Bech O, Berg K and Moan J. (1997). The pH dependency of protoporphyrin IX formation in cells incubated with 5-aminolevulinic acid. Cancer Lett 113(1-2):25-29.
Bunke A, Zerbe O, Schmid H, Burmeister G, Merkle H P and Gander B. (2000). Degradation mechanism and stability of 5-aminolevulinic acid. J Pharm Sci 89(10):1335-1341.
Calzavara-Pinton P G. (1995). Repetitive photodynamic therapy with topical delta-aminolaevulinic acid as an appropriate approach to the routine treatment of superficial non-melanoma skin tumours. J Photochem Photobiol B 29(1):53-57.
Collins P, Robinson D J, Stringer M R, Stables G I and Sheehan-Dare R A. (1997). The variable response of plaque psoriasis after a single treatment with topical 5-aminolaevulinic acid photodynamic therapy. Br J Dermatol 137(5):743-749.
de Blois A W, Grouls R J, Ackerman E W and Wijdeven W J. (2002). Development of a stable solution of 5-aminolaevulinic acid for intracutaneous injection in photodynamic therapy. Lasers Med Sci 17(3):208-215.
de Blois A W, Thissen M R, de Bruijn H S, Grouls R J, Dutrieux R P, Robinson D J, et al. (2001). In vivo pharmacokinetics of protoporphyrin IX accumulation following intracutaneous injection of 5-aminolevulinic acid. J Photochem Photobiol B 61(1-2):21-29.
Elfsson B, Wallin I, Eksborg S, Rudaeus K, Ros A M and Ehrsson H. (1999). Stability of 5-aminolevulinic acid in aqueous solution. Eur J Pharm Sci 7(2):87-91.
Fink-Puches R, Wolf P and Kerl H. (1997). Photodynamic therapy of superficial basal cell carcinoma by instillation of aminolevulinic acid and irradiation with visible light. Arch Dermatol 133(12):1494-1495.
Fritsch C, Homey B, Stahl W, Lehmann P, Ruzicka T and Sies H. (1998). Preferential relative porphyrin enrichment in solar keratoses upon topical application of delta-aminolevulinic acid methylester. Photochem Photobiol 68(2):218-221.
Harth Y, Hirshowitz B and Kaplan B. (1998). Modified topical photodynamic therapy of superficial skin tumors, utilizing aminolevulinic acid, penetration enhancers, red light, and hyperthermia. Dermatol Surg 24(7):723-726.
Henderson B W and Fingar V H. (1987). Relationship of tumor hypoxia and response to photodynamic treatment in an experimental mouse tumor. Cancer Res 47(12):3110-3114.
Herman M A, Webber J, Fromm D and Kessel D. (1998). Hemodynamic effects of 5-aminolevulinic acid in humans. J Photochem Photobiol B 43(1):61-65.
Hongcharu W, Taylor C R, Chang Y, Aghassi D, Suthamjariya K and Anderson R R. (2000). Topical ALA-photodynamic therapy for the treatment of acne vulgaris. J Invest Dermatol 115(2):183-192.
Jeffes E W, McCullough J L, Weinstein G D, Fergin P E, Nelson J S, Shull T F, et al. (1997). Photodynamic therapy of actinic keratosis with topical 5-aminolevulinic acid. A pilot dose-ranging study. Arch Dermatol 133(6):727-732.
Kennedy J C, Pottier R H and Pross D C. (1990). Photodynamic therapy with endogenous protoporphyrin IX: basic principles and present clinical experience. J Photochem Photobiol B 6(1-2):143-148.
Lin X X, Wang W, Wu S F, Yang C and Chang T S. (1997). Treatment of capillary vascular malformation (port-wine stains) with photochemotherapy. Plast Reconstr Surg 99(7):1826-1830.
Martin A, Tope W D, Grevelink J M, Starr J C, Fewkes J L, Flotte T J, et al. (1995). Lack of selectivity of protoporphyrin IX fluorescence for basal cell carcinoma after topical application of 5-aminolevulinic acid: implications for photodynamic treatment. Arch Dermatol Res 287(7):665-674.
Meyer W. (1996). Bemerkungen zur Eignung der Schweinehaut als biologisches Modell für die Haut des Menschen. Der Hautarzt 47(3):178-182.
Morgan J, Oseroff A R and Cheney R T. (2004). Expression of the peripheral benzodiazepine receptor is decreased in skin cancers in comparison with normal skin. Br J Dermatol 151(4):846-856.
Novo M, Huttmann G and Diddens H. (1996). Chemical instability of 5-aminolevulinic acid used in the fluorescence diagnosis of bladder tumours. J Photochem Photobiol B 34(2-3):143-148.
Rhodes L E, Tsoukas M M, Anderson R R and Kollias N. (1997). Iontophoretic delivery of ALA provides a quantitative model for ALA pharmacokinetics and PpIX phototoxicity in human skin. J Invest Dermatol 108(1):87-91.
Rick K, Sroka R, Stepp H, Kriegmair M, Huber R M, Jacob K, et al. (1997). Pharmacokinetics of 5-aminolevulinic acid-induced protoporphyrin IX in skin and blood. J Photochem Photobiol B 40(3):313-319.
Ruiz-Rodriguez R, Sanz-Sanchez T and Cordoba S. (2002). Photodynamic photorejuvenation. Dermatol Surg 28(8):742-744; discussion 744.
Svanberg K, Andersson T, Killander D, Wang I, Stenram U, Andersson-Engels S, et al. (1994). Photodynamic therapy of non-melanoma malignant tumours of the skin using topical delta-amino levulinic acid sensitization and laser irradiation. Br J Dermatol 130(6):743-751.
Thissen M R, de Blois M W, Robinson D J, de Bruijn H S, Dutrieux R P, Star W M, et al. (2002). PpIX fluorescence kinetics and increased skin damage after intracutaneous injection of 5-aminolevulinic acid and repeated illumination. J Invest Dermatol 118(2):239-245.
Wolf P, Fink-Puches R, Cerroni L and Kerl H. (1994). Photodynamic therapy for mycosis fungoides after topical photosensitization with 5-aminolevulinic acid. J Am Acad Dermatol 31(4):678-680.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method for treating a tissue disorder in a subject, the method comprising the steps of:
parenterally administering a solution of aminolevulinic acid (ALA) or a derivative thereof that is not greater than 1.0 percent by weight into a bodily area of the subject; and
administering high fluence red light to said bodily area to produce a phototoxic species in said local region,
thereby treating a tissue disorder in the subject.

2. A method for treating a tissue disorder in a subject, the method comprising the steps of:
parenterally administering a solution comprising about 1.0 percent or less than 1.0 percent by weight aminolevulinic acid (ALA) or a derivative thereof and at least one antinociceptive agent into a bodily area of the subject; and
administering high fluence red light to said bodily area to produce a phototoxic species in said local region,
thereby treating a tissue disorder in the subject.

3. A method for reduction of lipid rich cells in a bodily area of a subject, the method comprising the steps of:
providing a solution of aminolevulinic acid (ALA) or a derivative thereof that is not greater than 1.0 percent by weight to a bodily area comprising fat tissue of the subject; and
administering high fluence red light to said bodily area to produce a phototoxic species in said local region,
thereby reducing lipid rich cells in the subcutaneous tissue of a subject.

4. The method of claim 3, wherein the solution is provided by parenteral administration.

5. The method of claim 1, wherein the administering of the solution is by injection.

6. The method of claim 5, wherein the injection is intradermal injection, subcutaneous injection, intraperitoneal injection or intravenous injection.

7. The method of claim 1, wherein the tissue disorder is vascular malformation.

8. The method of claim 7, wherein the vascular malformation is a hemangioma, a Port wine stain, Sturge-weber Sd., or a secondary neovascularization.

9. The method of claim 1, wherein the tissue disorder is non-neoplastic.

10. The method of claim 1, wherein the tissue disorder is a tumor.

11. The method of claim 1, wherein the tissue disorder is an eccrine gland disorder.

12. The method of claim 11, wherein the eccrine gland disorder is hyperhidrosis, hidradenitis suppurativa, syringomas, or bromohydrosis.

13. The method of claim 1, wherein the wavelength of the red light is between about 620 and about 645 nm.

14. The method of claim 1, wherein the fluence of the red light is between about 100 and about 300 J/cm².

15. The method of claim 1, wherein the irradiance of the red light is between about 30 and about 200 mW/cm².

16. The method of claim 1, wherein the aminolevulinic acid derivative is an ester of 5-aminolevulinic acid.

17. The method of claim 1, wherein the solution is between about 0.015% and about 1.0% ALA.

18. The method of claim 1, wherein the solution is between about 0.0005% and about 1.0% ALA.

19. The method of claim 11, wherein the solution is about 0.06% ALA.

20. The method of claim 7, wherein the solution is about 0.25% ALA.

21. The method of claim 3, wherein the solution is less than or equal to about 0.016% ALA.

22. The method of claim 1, wherein the bodily area is a local subcutaneous or dermal region.

23. The method of claim 1, wherein the bodily area comprises dermis, or subcutis layer of skin.

24. The method of claim 1, wherein the bodily area is an internal body organ or tissue.

25. The method of claim 3, wherein the bodily area comprises a local subcutaneous region of the subject.

26. The method of claim 3, wherein the fat tissue is part of an internal organ or tissue.

27. The method of claim 3, wherein the fat tissue is part of the endovascular system.

28. The method of claim 27, wherein the fat tissue is a part of an atherosclerotic plaque of a blood vessel.

29. The method of claim 1, wherein the phototoxic species is a porphyrin.

30. The method of claim 29, wherein the porphyrin is protoporphyrin IX or a derivative thereof.

31. The method of claim 1, wherein the solution further comprises at least one antinociceptive agent.

32. The method of claim 2, wherein the at least one antinociceptive agent is an opioid analgesic.

33. The method of claim 2, wherein the at least one antinociceptive agent is a local anesthetic.

34. A pharmaceutical composition comprising about 1.0 percent by weight aminolevulinic acid (ALA) or a derivative thereof and at least one antinociceptive agent and a pharmaceutically acceptable carrier therefor.

35. The pharmaceutical composition of claim 34, wherein the at least one antinociceptive agent is an opioid analgesic.

36. The pharmaceutical composition of claim 34, wherein the at least one antinociceptive agent is a local anesthetic.

37. A packaged pharmaceutical comprising aminolevulinic acid (ALA) or a derivative thereof that is not greater than 1.0 percent by weight and instructions for use in accordance with the method of claim 1.

38. The packaged pharmaceutical of claim 37, further comprising at least one antinociceptive agent.

39. The packaged pharmaceutical of claim 38, wherein the at least one antinociceptive agent is an opioid analgesic.

40. The packaged pharmaceutical of claim 38, wherein the at least one antinociceptive agent is an anesthetic.

41. The method of claim 1, further comprising obtaining the ALA or derivative thereof.

42. The method of claim 1, wherein the bodily area is a local subcutaneous or dermal region of the subject.

43. The method of claim 1, wherein the bodily area excludes the epidermis.
